(19) United States
(12) Patent Application Publication
(10) Pub. No.: US 2015/0104512 A1
(43) Pub. Date: Apr. 16, 2015

(54) COATED TABLETS AND THE PRODUCTION THEREOF

(71) Applicant: Merck Patent GmbH, Darmstadt (DE)

(72) Inventors: Roberto Ognibene, Darmstadt (DE); Sandra Erika Bernhardt, Muehltal (DE); Melanie Mechthild Breidung, Babenhausen/Hergershausen (DE); Dieter Lubda, Bensheim (DE); Hans-Leonhard Ohrem, Jugenheim (DE)

(73) Assignee: Merck Patent GmbH, Darmstadt (DE)

(21) Appl. No.: 14/396,166

(22) PCT Filed: Apr. 2, 2013
(86) PCT No.: PCT/EP2013/000971

§ 371 Date: Oct. 22, 2014

(30) Foreign Application Priority Data
Apr. 27, 2012 (EP) 12002988.9
Sep. 11, 2012 (EP) 12006370.6

(51) Int. Cl. A61K 9/28; A61K 9/20; A61K 31/375

(52) U.S. Cl. CPC A61K 9/284; A61K 31/375; A61K 9/2018; A61K 9/2013; A61K 9/2893
USPC 424/465; 514/474; 427/2.21

(57) ABSTRACT

The present invention provides a rapidly disintegrating pharmaceutical formulation in the form of a coated tablet having increased mechanical strength, or hardness. The invention furthermore relates to a process for the production of the coated tablet and to the use of these formulations.

The present invention provides a rapidly disintegrating pharmaceutical formulation in the form of a coated tablet having increased mechanical strength, or hardness. The invention furthermore relates to a process for the production of the coated tablet and to the use of these formulations.

Coated tablets have been known for a long time. The coating serves, inter alia, for protection against disadvantageous physical and mechanical influences of the active-compound constituents present in the formulations. Adverse external influences of this type may be caused, for example, by UV light, oxygen or moisture, but also by mechanical load, such as impact and friction, with the result that active compounds and assistants may decompose or abrasion of the tablets means that the dosage is no longer guaranteed.

In principle, coating of tablets is carried out for the following reasons:
identification and recognition on the market and by customers due to specific colours,
taste masking for medicaments which have an unpleasant taste,
coating for delayed and/or targeted release (for example gastric juice-resistant),
improvement in the surface nature, giving rise to advantages in packaging and enabling the tablets to be swallowed better by the user.

According to experience, tablets can be protected against external influences by a suitable protective coating. Corresponding coatings may consist of a glaze, which may comprise sugar for taste improvement and may optionally be coloured. The latter may take place for aesthetic reasons in order to make the formulation in the form of tablets stand out on the market.

Apart from sugar-containing coatings, glazes which consist of a natural or synthetic polymer film which is not toxic to humans are also known. The latter can consist, for example, of gelatine, methylcellulose, polyvinylpyrrolidone, polyvidone acetate or other tolerated polymers which, depending on the intended site of action of the medicament, dissolve rapidly or only dissolve in a delayed manner in the stomach or small intestine. In order to improve the solubility of the coating, the polymers can be employed in combination with a small amount of a soluble component, so that on the one hand the film-forming properties of the polymer can be utilised and on the other hand it is ensured that the film or coating at least partially dissolves in the oral cavity in the presence of moisture and the tablet core thus becomes accessible to moisture and disintegrates rapidly as desired. Various ready-to-use solutions for the application of glazes or films to tablets are commercially available.

As so-called film tablets, tablets for the oral administration of medicaments, vitamins and in particular for fast-acting medicaments are frequently offered. Together with the choice of a favourable shape, the smooth surface forming due to the coating can make it easier to take the tablets, enabling them to be swallowed better. At the same time, the coating can, as already mentioned above, serve for taste masking for medicaments which have an unpleasant taste. As already indicated, it also contributes to increasing the impact strength and water resistance of the pressed tablet core. The improved impact strength is advantageous during machine packaging of the tablets, in particular if the tablets are packed in foils, so-called blisters. The coating prevents abrasion. Some particularly soft tablet cores can only be packaged after application of a suitable coating.

In addition, colouring or marking of the tablet coating can contribute to increasing recognition and preventing and avoiding confusion of tablets, which is essential, for example for elderly patients or those with dementia, in order to recognise the tablets having certain pharmaceutical active compounds. However, it can also serve for medicament safety through the use of specific colours. Such colours present in the coating can protect light-sensitive active compounds and assistants present in the tablet. Accordingly, corresponding tablet coatings offer protection for the substances present in the tablet core against external influences, such as light, heat, moisture, but also for the tablet itself against mechanical abrasion.

In order that tablets can be treated at all in a coating process of this type, the tablets must have corresponding strength and may only exhibit very low abrasion.

In recent years, there has been a corresponding constant increase in the popularity for a variety of areas of application of so-called ODTs (“oral dispersible tablets”), which on the one hand disintegrate rapidly in the mouth on contact with moisture (for example saliva), owing to additives present, such as, for example, disintegrants (superdisintegrants), polymers, starches, etc., but on the other hand must also meet the prerequisites if the tablet cores produced are to be provided with a coating.

Various mixtures, so-called ready-to-use assistant systems, are commercially available for this purpose for the production of orally rapidly disintegrating tablets. In general, they can only be used for the production of tablets having low hardnesses, where the low hardnesses are accompanied by relatively high abrasion. Corresponding ODTs are moisture-sensitive, meaning that they are not suitable for the aqueous coating methods which are usual in the pharmaceutical industry. Consequently, the production of coated tablets with these mixtures is not possible.

In addition, the low strength means that unacceptable abrasion can be generated in the coating equipment, which may be associated with considerable dust formation. Furthermore, high abrasion means that the tablets do not have a complete functional or aesthetic coating after coating on the surface. The abraded particles, which have been fixed to the surface by the coating material and stuck there again, may be clearly visible on the surface of tablets produced in this way. Furthermore, corresponding tablets may be considerably damaged by relatively large pieces chipping off or breaking off the surface, potentially causing bevels, edges, inscriptions present to be considerably damaged.

On the other hand, coated tablets do not just have advantages. The coating may be a hindrance if the administered tablets are intended to dissolve rapidly in the mouth directly after they have been taken. The coating may significantly extend the disintegration times, since the tablet core coating operation is usually associated with subsequent hardening. In addition, the active compounds present in the tablet core may interact with the coating ingredients during the coating operation and storage and may react with them with formation of undesired by-products.

The composition of the starting solution for the production of the coating must therefore be selected in a suitable manner so that the coating formed can at least partially dissolve or form cracks in order that the tablet core is able to dissolve or disintegrate rapidly due to the moisture in saliva. Nevertheless, the coating must be stable on storage, so that corresponding tablets do not decompose on storage at elevated atmospheric moisture contents or disintegrate on contact with small amounts of moisture. The latter would no longer guarantee the tablet dosage and taking by the patient would thus likewise no longer be possible.

In order on the one hand to ensure rapid disintegration in the mouth in spite of the tablet coating, but on the other hand also to be able to utilise the advantages of the coating for the stability of the tablets, developers have proposed various solutions.

EP 2 433 621 A1 proposes, for example, coatings with gaps in the coatings which are located in grooves and are generated during the production of the coating. However, it is disadvantageous that the production of the coating requires a special process and the latter cannot readily be carried out in existing apparatuses.

However, tablet abrasion is not only of importance during storage. In particular, it also plays a role during production of the desired coating, since it must be assumed that considerable tablet abrasion means that the active-compound content and the desired individual dose of the administered tablet can no longer be ensured. This may have severe consequences in the case of underdosing of the active compound due to excessive loss through abrasion, since an optimum dosage of the pharmaceutical active compound is not achieved. It could also result in overdosing of the active compound if abraded material is re-incorporated in the coating of undamaged tablets. It is vital that both are avoided. Premature decomposition or disintegration of OD tablets during storage due to moisture or during production of the coating would also change the properties of the tablets and result in non-reproducible production methods.

### OBJECT

The object of the present invention is therefore to provide a composition for the production of tablets which enables the production of tablet cores having high hardness and low abrasion which disintegrate rapidly in the mouth and which can easily be provided with a stabilising coating with virtually no change, but by means of which the disintegration time of the coated tablet changes as little as possible compared with that of the tablet core.

For this reason, the rapidly disintegrating core of a tablet of this type should, like known products, be simple to produce and should as far as possible be directly tabletable from the dry mixtures of the starting materials. The tablets produced should have high mechanical strength, so that they remain undamaged in the subsequent treatment, such as, for example, during production of the coating and during packaging, transport and, where appropriate, when pressed out of the packaging.

In particular, it is therefore an object of the present invention to provide a combination of hard tablet core having low abrasion and a suitable coating, which enables the production of uniformly coated tablets comprising active compound which have high hardnesses and disintegrate rapidly in the patient's mouth on administration. It is furthermore an object of the invention to provide a suitable process which is simple to carry out, by means of which coated tablets which still disintegrate rapidly in the mouth when taken on the rapidly disintegrating tablet cores with as little abrasion as possible with retention of the tablet properties are obtained.

### ACHIEVEMENT OF THE OBJECT

The present invention surprisingly enables the provision of pharmaceutical formulations in the form of a coated tablet which disintegrates rapidly in the presence of moisture and consists of a tablet core
a) which is obtainable from a homogenised, directly compressible co-mixture of spray-granulated mannitol and crosslinked croscarmellose-sodium and at least one pharmaceutical active compound or food supplement and additives,
   and
b) a coating which is applied in the form of an aqueous or water- and alcohol-containing solution and dried.

For the production of the tablet core of this pharmaceutical formulation, a co-mixture consisting of 90 to 95% by weight of mannitol and 3 to 7% by weight of croscarmellose-sodium as tablet disintegrant and optionally up to 1% by weight of magnesium stearate is used.

Corresponding tablets are highly suitable as so-called IR or FR tablets.

The co-mixture used for the production of the tablet core has a flow angle in the range from 33 to 38°, particle sizes in the range from 70 to 120 μm (Dᵥ₅₀; laser), a bulk density in the range from 0.55 to 0.65 g/ml and a tapped density in the range from 0.70 to 0.80 g/ml. The large BET surface area of the co-mixture in the range from 2.4 to 3.5 m²/g has a particularly advantageous effect. In accordance with the invention, the tablet core used may comprise a pharmaceutical active compound or food supplement in an amount of 0.1 to 50% by weight, based the weight of the tablet core. For the production of the rapidly disintegrating tablet according to the invention, the coating is applied in the form of a water- or water/ethanol-containing solution which, for the formation of the coating, comprises soluble film formers from the group polyvinylpyrrolidone, vinylpyrrolidone-vinyl acetate copolymer, polyvinyl acetate, hydroxypropylmethylcellulose, methacrylate copolymer or mixtures thereof. The coating solution may comprise further substances which have a pleasant effect in the mouth even during dissolution of the outer tablet coating. These can be one or more sugars from the group glucose, dextrose, fructose, lactose, maltose, xylose, sucrose, corn syrup, sorbitol, hexitolol, maltitol, xylitol and mannitol, optionally at least one polyalcohol selected from the group glycerol, polyethylene glycol and propylene glycol, and optionally at least one edible acid which is suitable for foods, from the group citric acid, malic acid, tartaric acid, fumaric acid, phosphoric acid, oxalic acid and ascorbic acid, and aroma oils and/or flavours and optionally a sweetener, such as, for example, sucralose, aspartame, acesulfame-K or the like.

In accordance with the invention, the coated tablets which disintegrate rapidly in the presence of moisture may comprise at least one active compound selected from the group atypical antipsychotics, antipsychotics, antidepressants, antihistamines, acetylcholinesterase inhibitors, analgesics, antipyretics, anticonvulsant, anticholinergic, antiemetics, benzodiazepines, corticosteroids, DDC inhibitors [carbidopa], dopamine receptor antagonists, monoamine oxidase inhibitors (MAOIs), non-benzodiazepine hypnotics, opioid analgesic [tramadol], proton pump inhibitors, triptans/serotonin agonists, NSAIDs and SSRIs.

It has proven particularly advantageous for the pharmaceutical formulations prepared in this way for the uncoated tablet core to have low abrasion of less than 0.50%, based on the weight, since this enables the desired coatings to be applied very uniformly and for the tablets produced to have a very uniform surface.

While it was hitherto regarded as impossible to provide tablets which disintegrate rapidly in the presence of moisture with a coating by means of water-containing solutions, the process according to the invention using the tablet cores described enables corresponding pharmaceutical formulations to be prepared by warming the tablet cores, which have been produced in advance using the above-mentioned co-mixture, to an elevated temperature in a coating drum with mixing and producing the coating by spraying the low-viscosity coating solution onto the tablet cores and drying it at elevated temperature. The tablet cores are warmed here to a temperature in the range from 35 at 60° C. before the spraying-on of the coating solution. Particularly good results are achieved if the tablet cores are warmed to a temperature in the range from 40 to 55° C. and the tablets are dried for 10 to 20 minutes after the spraying-on of the coating solution.

### DETAILED DESCRIPTION OF THE INVENTION

As already described, tablets which rapidly disintegrate rapidly in the mouth are becoming ever more popular for oral administration. Tablets of this type should disintegrate in the mouth within less than 90 seconds, preferably less than 60 seconds, particularly preferably in a time of not more than 30 seconds, in the oral cavity, where they should produce a pleasant mouth feel and should taste good. According to the most recent requirements, a rapidly disintegrating tablet of this type should have a disintegration time of less than 18 seconds. This in most cases presents the developer (person skilled in the art) with major and in some cases insoluble challenges.

The rapidly disintegrating core of a tablet of this type should, like known products, be simple to produce and should as far as possible be directly tabletable from the dry mixes of the starting materials. The tablets produced should have high mechanical strength, so that they remain undamaged in the subsequent treatment during packaging, transport and, where appropriate, when pressed out of the packaging.

In general, it is therefore desirable for the abrasion of the tablet core during the coating process to be less than 0.2 to 0.4% by weight.

A very wide variety of ready-to-use mixtures from which rapidly disintegrating tablets can be produced together with the desired active compound are per se commercially available for the production of rapidly disintegrating tablets. A very wide variety of tableting assistants which are used in the pharmaceutical industry may be present therein, but these are always combined with a substance which reacts with the moisture in the mouth and result in swelling and bursting, i.e. in disintegration of the tablet. Vehicles which can be employed in corresponding ready-to-use mixtures for the production of the active-compound-containing tablets are preferably hydroxyl-containing natural substances. Such carrier substances, frequently also referred to as excipients, are polyols, such as mannitol, xylitol, sorbitol, erythritol, but also lactose or starch and derivatives thereof or other neutral substances which have no effect on the activity of the pharmaceutical active compound and, on pressing of the tablets, result in sufficiently hard tablets. However, it is not readily possible to provide the tablet cores produced which disintegrate rapidly in the presence of moisture with a desired coating after pressing with the aid of water-containing compositions since the tablet core may change even in the presence of only small amounts of liquid. Possible abrasion may also have an adverse effect on the quality of the coating.

In order to produce film tablets from the rapidly disintegrating tablet cores produced, the following problems must generally be expected, compared with a “normal” tablet:
Significantly longer disintegration times (associated with hardening after the coating operation) and consequently an undesired delay/extension of the release of the active compound
Interactions of the medicament with the coating layer, consequently the formation of undesired by-products.

Experiments have shown that rapidly disintegrating tablets having high hardnesses can be produced from ready-to-use mixtures, as described in the application WO 2009/152922 A1, even at a low pressing pressure, as desired. These ready-to-use mixtures are commercially available from Merck (Darmstadt, Germany) under the trade name Parteck ODT®. The product has a flow angle in the range from 33 to 38° with particle sizes in the range from 70 to 120 μm (D₅₀; laser). The bulk density is in the range from 0.55 to 0.65 g/ml with a tapped density of 0.70 to 0.80 g/ml. At the same time, this ready-to-use mixture has a high BET surface area in the range from 2.4 to 3.5 m²/g. This product consists of 90 to 95% by weight of mannitol and 3 to 7% by weight of croscarmellose-sodium as tablet disintegrant.

Orally rapidly disintegrating tablets having low friabilities and good properties during packaging and storage can be produced by direct pressing from a mixture of Parteck ODT® with up to 50% by weight of an active compound and optionally 1% by weight of magnesium stearate or sodium stearyl fumarate or another lubricant or glidant usually used, based on the total weight of the mixture.

Other ready-to-use mixtures are marketed, for example, under the names Ludiflash®, Perlitol® Flash, Pharmaburst® 500 or Prosolv® ODT. Further mixtures and the use thereof are described in the review by B. G. Prajapati and N. Ratnakar, [Int. J. of. PharmTech Research, Vol. 1, No. 3, 790-798, (2009)]. Of the said ready-to-use mixtures, Parteck® ODT, Ludiflash®, and Perlitol® Flash, in particular, are based on mannitol as the main ingredient or carrier material, while Prosolv® ODT consists of a mixture of microcrystalline cellulose, colloidal silicon dioxide, in each case 30-40% of mannitol and fructose, and crospovidone as tablet disintegrant.

The ready-to-use mixture obtainable from BASF (Germany) under the trade name Ludiflash® consists of D-mannitol, crospovidone, polyvinyl acetate and small amounts of povidone. Polyvinyl acetate is introduced into the formulation as Kollicat® SR 30 D, which consists of a polyvinyl acetate dispersion which is stabilised by povidone. This composition is a white, free-flowing powder which has a flow angle of about 38° and has a particle distribution as follows:

| | |
|---|---|
| >0.400 mm | max. 20% |
| <0.200 mm | max. 90%, min. 45% |
| <0.063 mm | max. 45%, min. 15% |
| bulk density: | 0.40-0.52 g/ml. |

The Perlitol® Flash ready-to-use mixture from Roquette consists of a spray-dried composition comprising 80% of mannitol and 20% of corn starch.

The ready-to-use mixtures marketed by SPI Pharma under the trade name Pharmaburst® again 73-94% of a polyol combination which consists, according to the company information, of mannitol and sorbitol, and comprises tablet disintegrant and glidant.

F-Melt® Type C or M (Fuji Chemical Industry, Co., Ltd.) again comprises about 65% by weight of mannitol, xylitol, microcrystalline cellulose, crospovidone and other additives.

Pharmatrans SANAQ AG offers OroCell® 200 & 400 as further ready-to-use mixtures having a high mannitol content. The mixtures have a mannitol content of 90% and differ in that the first mixture has an average particle diameter of <315 μm and the second has an average particle diameter of <500 μm.

The ready-to-use mixture from Baker with the name PanExcea® ODT MC200G consists of 75% of mannitol and 25% of calcium silicate.

Comparative experiments have now shown that, in particular, corresponding ready-to-use mixtures which comprise a high content of spray-dried, granulated mannitol as carrier material can be pressed with a low pressing pressure to give tablets having relatively high hardnesses which at the same time have comparatively low friabilities in the test. The suitable combination of the mannitol grade and the tablet disintegrant present in the ready-to-use mixture appears to be important in this connection. A high proportion of spray-dried, granulated mannitol with a matched particle-size distribution determines on the one hand the flowability of the mixture predominantly through the properties of the mannitol, which is in turn essential for a problem-free tableting process. In particular, the compressibility of all components present in the ready-to-use mixture is also important for tableting, enabling the lowest possible pressing pressure to be used. Unexpectedly, it has been found that corresponding mannitol-containing compositions, but in particular compositions which simultaneously comprise croscarmellose-sodium as disintegrant, such as, for example, the ready-to-use mixture Parteck ODT, can be pressed even at low pressure to give hard tablets, where the latter simultaneously exhibit a low friability of less than 0.35%, whereas it is in some cases only possible to determine a friability of about 1-2% for comparable compositions after the same processing.

After pressing, the tablet cores produced, which have very high mechanical stability and fast disintegration, can surprisingly be provided in the next process step, in spite of the tablet disintegrant present, with a coating of a water- or water- and ethanol-containing composition in a suitable coating apparatus without significantly changing the disintegration properties of the tablet treated in this way, so that the active-compound-containing tablet core is subsequently protected against external influences. This possibility is all the more surprising as rapidly disintegrating tablets known to date, in which a coating appeared necessary for protection of the active compounds present, have only been produced from coated, active-compound-containing particles, as described, for example, in US 2011/0129530 A1, since the coated, active-compound-containing particles therein are mixed with further filler and/or binder, a tablet disintegrant and a glidant, such as magnesium stearate, and pressed to give rapidly disintegrating tablets.

The idea that orally rapidly disintegrating tablets, owing to their sensitivity to moisture, cannot have an outer coating, but instead can only be produced by pressing the starting mixture, is also reflected in the pharmacopoeias. For example, the European Pharmacopoeia describes such tablets as uncoated tablets which disintegrate rapidly in the mouth before they are swallowed. (Ph. Eur., 2005).

By contrast, it has been found, in particular on use of the ready-to-use mixtures described in the application WO 2009/152922 A1, which are marketed under the trade name Parteck® ODT, that the tablet cores can, surprisingly, be coated with water-containing compositions after tableting and it is possible to produce tablets coated in this way which have adequate or even increased strength and very low abrasion. After application of the coating, the tablets still have short disintegration times. Under certain conditions, the disintegration times in the presence of moisture, such as in the mouth, may even be reduced.

The particular feature of the tablet matrix produced from Parteck® ODT is that, in spite of the coating, fast disintegration of the tablets in the mouth is achieved at the same time as sufficiently high hardnesses, and that this is surprisingly also the case after a coating process using water- or water/alcohol-containing compositions, although it would actually be assumed that the coating applied would considerably extend the disintegration time. In this connection, it is particularly surprising that, depending on the composition employed for production of the coating, the disintegration time of the tablet may even be shortened with increasing hardness and the tablets obtained can be formulated as so-called IR tablets or FR tablets, which disintegrate rapidly in the mouth, and the active compounds can be taken up directly via the oral mucous membrane.

The pharmacist, but also the formulator of tablet-form food supplements, is thus given the possibility of producing corresponding, rapidly disintegrating, coated tablets. The coating enables all the disadvantages enumerated above to be avoided and the corresponding advantages to be achieved.

For the manufacturer, the coating of tablets is of particular importance, since, for example, the active compounds present are thereby protected against external influences and, in particular, the shelf life of the tablets, which may be packaged in tubs or blisters, can be improved. However, it can also serve to improve the outer appearance of the tablets for the user and to increase the recognisability.

Corresponding rapidly disintegrating tablets are particularly advantageous for formulations in which the following active compounds may be present: atypical antipsychotics, antipsychotics, antidepressants, antihistamines, acetylcholinesterase inhibitors, analgesics, antipyretics, anticonvulsant, anticholinergic, antiemetics, benzodiazepines, corticosteroids, DDC inhibitors [carbidopa], dopamine receptor antagonists, monoamine oxidase inhibitors (MAOIs), non-benzodiazepine hypnotics, opioid analgesic [tramadol], proton pump inhibitors, triptans/serotonin agonists, NSAIDs and SSRIs.

Although it would have been expected that the coating step has an adverse effect on the properties of the tablet core owing to the use of moisture-containing composition, it has been found on use of tablet cores produced using Parteck® ODT that the coating process can be carried out without adversely affecting the disintegration properties. This result was found both for placebo formulations and also for active-compound-containing tablet cores.

By contrast, commercially available ready-to-use mixtures subjected to comparative testing which likewise have a high mannitol content exhibited significantly worse behaviour during the coating operation and in the subsequent hardness and friability testing. Thus, a significant increase in the disintegration time of up to 10 to 13-fold after the coating process was found for comparative products. These tablet cores are thus no longer suitable or only of restricted suitability for rapidly disintegrating applications. In addition, considerable abrasion or the breaking-off of tablet constituents (bevel) was observed for some of these products during the coating operation.

As already mentioned above, a very wide variety of premixed compositions are commercially available to the pharmacist or formulator as ready-to-use coating systems for the production of tablet coatings in the form of glazes or films.

They are predominantly suitable for water-containing coating systems. In particular cases, they are formulations or coating systems which can also be used for coatings based on water/ethanol. Such ready-to-use coating systems are offered by various manufacturers and are readily commercially available to the user. Depending on the desired use, the compositions can, as already indicated, comprise various stable coloured pigments or water-soluble dyes which are not toxic to humans for the colour design of the outer appearance of the coated tablets. Tolerated pigments which may be present are, for example, Candurin pigments. These are pigments which are approved as food colours and as pharmaceutical assistants and consist of natural silicates and naturally occurring oxides, such as titanium dioxide and/or iron oxide, or are mineral pearlescent pigments. On addition of the pigments, it must be ensured that the properties of the coating are influenced as little as possible. In the present case, the solubility on contact with moisture, in particular with saliva, must not be impaired in order to retain the rapid solubility of the tablet core.

The composition for the production of the coating may comprise water-soluble polymers selected from the group carboxymethylcellulose, carboxyvinyl polymers, high-amylose starch, hydroxyethylcellulose, hydroxypropylcellulose, hydroxypropylmethylcellulose (hypromellose), methyl methacrylate copolymers, polyacrylic acid, polyvinyl alcohol, polyvinylpyrrolidones, pullulan, sodium alginate and mixtures thereof.

Coatings are preferably produced from compositions which comprise, as film formers, polymers selected from the group polyvinylpyrrolidone, vinylpyrrolidone-vinyl acetate copolymer, polyvinyl acetate, hydroxypropylmethylcellulose and methacrylate copolymer or mixtures thereof.

Compositions for the production of the film coatings may furthermore comprise sugars selected from the group glucose, dextrose, fructose, lactose, maltose, xylose, sucrose, corn syrup, sorbitol, hexitol, maltitol, xylitol, mannitol and mixtures thereof.

At least one polyalcohol selected from the group glycerol, polyethylene glycol (for example macrogol), propylene glycol, or mixtures thereof, may also be present therein.

A composition of this type for the production of a coating on the tablet cores may comprise an edible acid which is suitable for foods, from the group citric acid, malic acid, tartaric acid, fumaric acid, phosphoric acid, oxalic acid and ascorbic acid or combinations of these acids.

In addition, the film-forming composition may comprise aroma oils and flavours which have a pleasant effect even during dissolution of the outer tablet coating and prevent an unpleasant taste in the mouth. Additives of this type can be selected from the group eucolyptol, menthol, thymol, verbenone, rosemary verbenone, eugenol, geraniol and others. However, it is also possible to use corresponding mixtures or other pleasant-tasting additives including sugars therein.

In addition, the mixtures for the production of the coatings may comprise further additives which have an advantageous effect on film formation, processability and the outer appearance of the finished tablet.

Correspondingly, tablets may be provided, for example, with coatings which consist of constituents such as hypromellose, macrogol 400 [mixture of linear polymers having the general formula H—(OCH₂—CH₂)ₙ—OH, with an average molecular weight of 400], talc (magnesium silicate hydrate), titanium dioxide (E171) and optionally iron(III) hydroxide oxide×H₂O (E172).

It is essential that the tablet coating dissolves in a few seconds in the presence of moisture, in particular saliva. Suitable coatings have dissolved in less than 90, preferably less than 60 seconds; where particularly suitable coatings have dissolved in a time of not longer than 10 seconds. During dissolution, these coatings should not produce a tacky feel in the mouth and should taste pleasant. The dissolution operation should preferably take place in such a way that the moisture can come into contact with the underlying tablet core as soon as the coating starts to dissolve, so that the core begins to disintegrate at the same time as the coating.

As already mentioned above, a very wide variety of ready-to-use mixtures for the production of tablet coatings are commercially available. Ready-to-use mixtures of this type are known to the person skilled in the art. When selecting a suitable composition, aqueous compositions and those in which, besides water as solvent, other solvents which are suitable for tablet production can also be added are available. Systems for aqueous solutions or dispersions are preferably used for this purpose or those which comprise merely a tolerated, readily volatile alcohol, such as ethanol, in water. The ready-to-use mixtures are a solids mixture which are dispersed or dissolved in water or a water/alcohol-containing solvent by the user before the coating process.

In the presence of rapidly disintegrating, moisture-sensitive tablet cores, it is essential that a continuous, thin protective film can form on the surface in a very short time by means of the finished water-containing composition for the coating, where the moisture concentration at the surface of the core is kept as low as possible. This can be achieved on the one hand only by means of low-viscosity compositions which spread out over the surface or immediately form a film from the impacting droplets on spraying of small amounts of coating solution, on the other hand the moisture concentration at the surface can only be kept low at elevated temperature. Accordingly, the rapidly disintegrating tablet cores must meet high requirements for the coating operation in order to avoid being damaged and/or destroyed owing to the high mechanical and thermal load during the coating process.

One of the most important properties of the tablet cores used in accordance with the invention consists in that they have low abrasion of <0.50%. Tablets having an abrasion of <0.2-0.3% can usually also be employed to a limited extent for coating by the person skilled in the art. However, excessively soft tablet cores cannot withstand the high mechanical loads in a coating drum with mixing paddles since they are already damaged during the heating time at the beginning owing to the lack of protection by a coating layer.

The experiments on the production of coated, rapidly disintegrating tablets have in addition shown that excessively soft tablets and tablets having high proportions of cellulose derivatives, and also ODT systems tend to swell already during the first application of the coating in an amount of 1-5% by weight of the total amount of coating applied. The assistants employed in the tablets attract the water, and the tablets already begin to disintegrate in the coating drum. Under these circumstances, further coating is no longer possible.

By contrast, coating experiments on tablet cores using a ready-to-use mixture for the production of rapidly disintegrating tablets, as have been produced in WO 2009/152922 A1, have now refuted the opinion previously regarded as generally valid. Through the use of the composition prepared in accordance with WO 2009/152922 A1 (Parteck® ODT), a coating or film can be applied to rapidly disintegrating tablets using an aqueous composition without this being accompanied by a significant extension of the disintegration time.

In spite of the coating layer applied and a simultaneous increase in the tablet hardness, the disintegration times of the film tablets obtained have only changed slightly (not significantly) compared with those of the original tablet cores.

For example, tablet cores produced from placebo formulations and treated with a ready-to-use mixture as coating system (Opadry 200 White, marketed by Colorcon). Amazingly, the disintegration time of corresponding Parteck ODT film tablets dropped by 3 seconds to 53 seconds compared with the disintegration time of the tablet cores, which disintegrate within an average of 56 seconds. The result is surprising since the hardness of the uncoated tablet cores increases from 51 N to 108 N after coating. In spite of the considerably higher hardness, a decrease in the disintegration time can surprisingly be demonstrated. The Opadry White coating system used is an aqueous composition in which the film formation takes place by the polyvinyl alcohol present. Depending on the concentration, the viscosity can be set low.

In comparison, tablets were produced using the Ludiflash® ready-to-use mixture described above and coated under the same conditions with the same coating system (Opadry® 200 White). Although the ready-to-use mixture used is likewise a composition having a high mannitol content, the disintegration time in the presence of moisture increases to an unexpectedly great extent after the tablet cores have been provided with an Opadry® film. Whereas the tablet cores have a short disintegration time, the coated tablets exhibit an increase in the disintegration time by 461 seconds to an average of 501 seconds. In this case too, an increase in the tablet hardness is noted; more precisely, the hardness increases from 44 N of the tablet cores to 93 N of the film tablets.

If tablet cores of the two placebo formulations are treated and coated with the Biogrund coating system (ReadiLycoat), a similar behaviour of the two different ODT recipes is apparent.

ReadiLycoat® is an aqueous coating system in which the coating is formed by a starch-based polymer.

The disintegration time of the film tablets produced using Parteck® ODT is on average 79 seconds with a hardness of 90 N. It has only increased by 23 seconds compared with the disintegration time of the original tablet cores, from 56 seconds originally with a hardness of 51 N.

By contrast, the film tablets with Ludiflash® as basis have a very long disintegration time of on average 400 seconds with a hardness of 93 N. It has thus increased by 360 seconds from a disintegration time of the tablet cores of 40 seconds with a hardness of 44 N.

Even if active-compound-containing tablet cores based on Parteck® ODT and Ludiflash® are produced and treated with the aqueous coating systems in a corresponding manner, the same picture arises. For these experiments, verum formulations having a proportion of 20% of ascorbic acid per individual dose were used.

Also under these conditions, the increase in the disintegration time of the film tablets obtained from tablet cores in which Parteck® ODT has been used is significantly smaller than when Ludiflash® has been employed for the production of the tablet cores. At the same time, the increase in the hardness of the coated tablets comprising Parteck® ODT is significantly greater than when Ludiflash® has been employed for the production of the tablet cores. The latter only exhibit a very small increase in the tablet hardnesses after coating.

The tablet cores of the Parteck ODT recipe have a disintegration time of 28 seconds, with a hardness of 54 N. After coating with the Colorcon coating system (Opadry® 200 White), the disintegration time of the film tablets increases by 27 seconds to an average of 55 seconds (hardness of 117 N), and, after coating with the Biogrund coating system (ReadiLycoat®), an increase in disintegration from 46 seconds to 74 seconds (hardness of 93 N) is noted.

By contrast, the tablet cores of the Ludiflash recipe have a disintegration time of 22 seconds (hardness of 45 N), which increases drastically after coating. The film tablets thus have a disintegration time of 309 seconds with a hardness of only 62 N after coating with the Colorcon coating system, an increase in the disintegration time of 287 seconds, and the film tablets with the Biogrund coating system have a disintegration time of 175 seconds with a hardness of only 59 N and again here an increase in the disintegration time of 153 seconds.

In addition, the film tablets produced using Parteck® ODT exhibit virtually no damage and have a flat and smooth surface, since the tablet cores employed have the best prerequisites for coating. They have a hardness in the region of 50 N in order to ensure a fast disintegration time, but at the same time have adequate breaking strength (mechanical stability) in order to withstand the loads during coating, and especially during the heating phase. The placebo tablet cores have an abrasion of 0.37% with an average hardness of 51 N, and the verum tablet cores have an abrasion of 0.45% with an average hardness of 54 N.

The placebo tablet cores based on Ludiflash have an abrasion of 0.36% with a tablet hardness of on average 44 N. These are good starting prerequisites for coating. Correspondingly, the coated tablets, i.e. the film tablets obtained, have a smooth and flat surface.

However, the verum tablet cores based on Ludiflash® exhibit very high abrasion of 14.71% at an average hardness of 45 N. In some cases, the tablet cores also exhibited capping during the abrasion test. These poor tablet properties also have an adverse effect on the coating operation. The tablets cannot withstand the high mechanical loads during heating. In many tablets, the edges have crumbled away at the junction with the body. These tablets thus lose weight, and it is not possible to determine the amount applied after coating, since the individual weight of the film tablets has reduced owing to the fractures, in contrast to the original tablet cores. In addition, the film tablets have clear fracturing at the sides.

The experiments have shown that, surprisingly, ODT tablets can be coated using aqueous coating systems. The experiments have shown that the tablet cores which can be used for coating cannot be of any desired composition.

Thus, a significant increase in the disintegration time of the film tablets can be expected in the case of conventional ODT systems, such as, for example, using Ludiflash® for the production of the tablet cores, compared with the original tablet cores. This has the consequence that rapid release of the active compound or rapid disintegration of the tablet inside the oral cavity no longer occurs, as is actually desired for ODT systems and prescribed for such formulations.

During the processing, in particular the coating, of Parteck® ODT, only a small increase in the disintegration time is observed, although an enormous increase in the hardness is noted in comparison between the original tablet core and the film tablet produced therefrom. The film tablets have only disintegration times of 53 to 79 seconds, in spite of the protective coating layer, so that they, even with coating, rapid disintegration and thus also rapid release of the active compound is ensured. A further advantage is the enormous strength of the film tablets, making them additionally resistant to mechanical loads, which occur, for example, during packaging and transport.

However, it is not only on use of purely aqueous coating systems that the use of Parteck® ODT proves advantageous for the production of orally rapidly disintegrating film tablets. Also if coating formulations which comprises an ethanol/water mixture as solvent are used for the production of corresponding coatings.

The experiments have shown that in all formulations using Parteck® ODT, irrespective of whether they comprise active compound or are placebos, a surprising and significant decrease in the disintegration time of the film tablets is observed compared with that of the tablet cores employed, at the same time as an increase in the hardness by 40-75 N after coating with a coating system which comprises an ethanol/water mixture as solvent.

Depending on the composition of the coating system, a small to considerable decrease in the disintegration time of the tablets after application of the coating is, surprisingly, observed. The reduction in the disintegration time varies between 1 second (−4%, in illustrative Processing Example No. 11) up to a decrease of 64 seconds (−49% in Processing Examples No. 5 and No. 6).

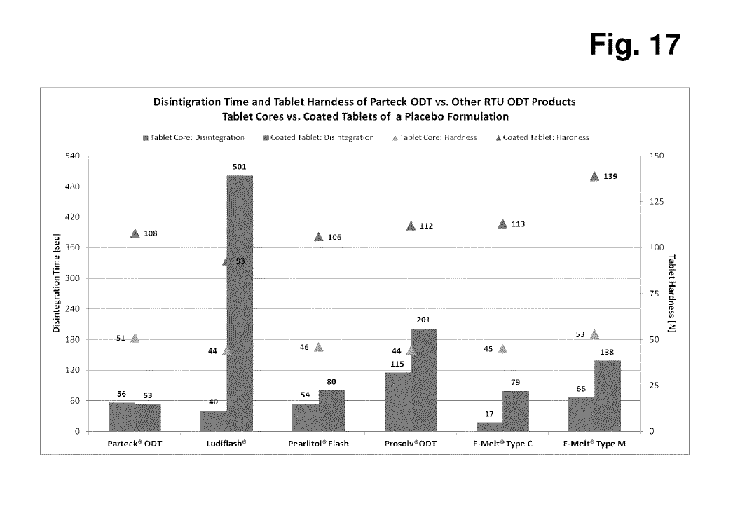

These results are particularly surprising since they cannot be understood in the same way with comparable, commercially available ready-to-use mixtures for the production of rapidly disintegrating tablets. In FIG. 17, various commercially available mixtures are compared with one another by on the one hand plotting the disintegration times and hardnesses of the tablet cores of different ready-to-use mixtures and on the other hand plotting the hardnesses and disintegration times of the corresponding coated tablet cores, where in each case a coating having the same composition was applied to the different tablet cores under the same conditions. In all cases in which the tablet cores consist of ready-to-use mixtures of other manufacturers, the hardness and disintegration time increase after application of the coating. By contrast, these values drop for tablet cores according to the invention (produced using Parteck® ODT.

For example, comparative experiments were carried out under the same conditions using the comparable ready-to-use composition Ludiflash® already mentioned above. Here, an extension of the disintegration time of the film tablets compared with that for the untreated tablet cores is determined in the case of all recipes prepared, irrespective of whether it is a placebo or active-compound-containing tablet formulation. In addition, virtually no increase in hardness is observed for active-compound-containing tablet formulations after the tablets have been provided with the coating (45 N to 46 N), but at the same time the disintegration time increases by on average 9 seconds.

Accordingly, Parteck® ODT for the production of the tablet cores is also particularly suitable for the production of rapidly disintegrating coated tablets on use of an ethanol/water-containing coating composition.

The present description enables the person skilled in the art to apply the invention comprehensively. Even without further comments, it is therefore assumed that a person skilled in the art will be able to utilise the above description in the broadest scope.

Should anything be unclear, it goes without saying that the publications and patent literature cited should be consulted. Accordingly, these documents are regarded as part of the disclosure content of the present description. This applies, in particular, to the Patent Application WO 2009/152922A1, in which the preparation of the ready-to-use mixture Parteck® ODT is described in greater detail.

For better understanding of the invention, examples and comparative examples which are within the scope of protection or illustrate the advantages of the invention are given below. These examples also serve to illustrate possible variants. Owing to the general validity of the inventive principle described regarding the use of the tablet mixtures and coating systems, the examples given are not suitable for reducing the scope of protection of the present application to these alone.

Furthermore, it goes without saying to the person skilled in the art that, both in the examples given and also in the remainder of the description, the component amounts present in the compositions always add up only to 100% by weight or mol %, based on the composition as a whole, and cannot exceed this, even if higher values could arise from the percent ranges indicated. This also applies to volume percent data. Unless indicated otherwise, % data are therefore taken to mean % by weight or mol %, with the exception of ratios, which are shown in volume figures.

The temperatures given in the examples and description and in the claims are always in ° C.

### EXAMPLES

### Equipment Used

1. Korsch PH230 rotary tablet press (KORSCH AG, Berlin, Germany)
   14 punch pairs: Ø11 mm, biconvex, R14.5, punch No. 05/11
   PMA3 evaluation system (Pharmapress measuring and analysis system) from Korsch
2. RRM ELTE 650 drum hoop mixer from Engelsmann (J. Engelsmann AG, Ludwigshafen, Germany)
   use of 50 l mixing drum, with no chicanes or mixing aids
3. O'Hara Labcoat IIX coater (Ohara Technologies, Richmond Hill, Canada)
   small drum (d=15 inches) fitted with a nozzle
   two-component nozzle from Schlick, diameter of nozzle insert: 0.7 mm
4. IKA stirrer (IKA®-Werke GmbH & CO. KG, Staufen, Germany)
   preparation of coating suspension with a propeller stirrer Ø about 8 cm
5. ERWEKA TBH 30 MD (ERWEKA® GmbH, Heusenstamm, Germany)
   determination of height, diameter and hardness in-process and after 1 day (n=20 tablets)
   calculation of the average values from the 20 measurements
6. METTLER AT 201 analytical balance (Mettler-Toledo GmbH, Giessen, Germany)
   working range: 0.05 to 200 g
   determination of the individual tablet weight after 1 day or determination of the individual film-tablet weight (n=20 tablets or film tablets)
   calculation of the average values from the weighing of the 20 tablets/film tablets
7. Tablet abrasion on ERWEKA TA 420 friability tester (ERWEKA® GmbH, Heusenstamm, Germany)
   instrument parameters and performance of the measurements in accordance with Ph. Eur. 7th Edition “Friability of uncoated tablets”(use of a Roch drum)
   sample weight in abrasion test:
      tablet weight≦650 mg→total weight about 6.5 g, corresponds to 17 tablets@400 mg=6.8 g
8. ‘disi’ 4-tablet disintegration-time tester: DISI automatic disintegration tester, Pharmatron Dr. Schleuniger (Pharmatron AG, CH-3600 Thun, Switzerland)
   determination of the disintegration time of the tablets after 1 day and the disintegration time of the film tablets (n=6 tablets)
   determination in 800 ml of deionised water at 37° C.±2K
   instrument parameters and procedure in accordance with Ph. Eur. 7th Edition “Disintegration time of tablets and capsules”, test A: tablets and capsules of normal size

General Process Description of the Preparation

A. Preparation of the Ready-to-Use Mixture

Material to be Tableted:

Parteck ODT® (Article No.: 1.0490.9050, manufacturer: Merck KGaA, Darmstadt, Germany)

or

Ludiflash® (Article No.: 56513304, manufacturer: BASF, Ludwigshafen, Germany)

is in each case introduced into a 50 l mixing drum together with ascorbic acid (Article No.: 83568.290, manufacturer: BDH Prolabo chemicals—The Chemical Brand from VWR International, Leuven, Belgium) and subsequently homogenised in the drum hoop mixer (speed 7) for 10 minutes.

The magnesium stearate component (Parteck® LUB MST, vegetable, Article No.: 1.00663.9020, manufacturer: Merck KGaA, Darmstadt, Germany) is sieved onto the resultant mixture through a 250 μm laboratory sieve and homogenised in the drum hoop mixer at speed 7 for a further 10 minutes.

For the preparation of the placebo mixture, the material to be tableted (Parteck® ODT or. Ludiflash®) is mixed directly with the magnesium stearate component (vegetable, Parteck® LUB MST) and sieved through a 250 μm laboratory sieve. The substances are subsequently introduced into a 50 l mixing drum and homogenised in the drum hoop mixer at speed 7 for 10 minutes.

B. Production of the Tablets

The four different mixtures are pressed in the PH230 rotary tablet press at different pressures. The aim here is to set the pressure so that a tablet hardness of 50 N is achieved. For this purpose, at least 100 tablets are taken at four points in time during the tableting as samples for evaluation of the pressing data/pharmaceutical formulation characteristics.

C. Coating Production

90% of the intended amount of water are initially introduced, and the base coat comprising Opadry™ 200 White (Article No.: 200F280000, manufacturer: Colorcon GmbH, Idstein, Germany)

or

Aqua Polish® Clear (Article No.: MY6128, manufacturer: BIOGRUND GmbH, Hünstetten, Germany)

is added in portions with stirring.

The base coat is subsequently stirred until a homogeneous, smooth coating material has formed (within about 30-45 minutes). The pigments are incorporated into the finished base coat with slow stirring. When the pigments are uniformly distributed in the coating material, the coating production is complete.

D. Production of the Film Tablets

The tablet cores are coated in the O'Hara Labcoat with the 15″ drum (small drum) and a nozzle (Schlick two-component nozzle, nozzle insert 0.7 mm). The prespecified amount of tablet cores is weighed out into the drum and de-dusted and pre-warmed before commencing the spraying. After the heating phase, the corresponding amount of coating material is sprayed onto the tablets. Throughout the entire coating operation, the coating suspension is continuously stirred in order to prevent the pigments from settling out.

In addition, samples are taken after a coating application rate of 10, 20, 30, 40, 50, 60, 70, 80, 90 and 100% for later visual assessment and evaluation of the physical properties. After the spraying, the film tablets are dried in the drum up to a certain exhaust air temperature and then packed in double PE bags and in a tin can.

During the production process, the following parameters are checked and recorded:

1. Feed and exhaust air temperature in ° C. during the heating

2. Batch size (amount of FITABK used and amount of coating applied in g)

3. Feed and exhaust air temperature in ° C. during the spraying operating

4. Spray rate in g/min

5. Spray pressure in bar

6. Nozzle diameter in mm

7. Feed and exhaust air temperature in ° C. during the drying

E. Process Parameters for the Production and Coating of the Tablet Cores

| | |
|---|---|
| Parameter | Setting/value |

Preparation of the ready-to-use mixture

| Mesh width of hand sieve for | 250 μm | |
|---|---|---|
| lubricant | (magnesium stearate) | |
| Mixing time | 10 min for placebo formulations | |
| | 2 × 10 min for verum formulations | |
| Mixing speed | 7 | rpm |

Tableting

| | | |
|---|---|---|
| Machine performance | 50 | rpm |
| Pressing force | 5.1-14.5 | kN* |
| Fill depth | 5.6-6.5 | mm* |
| Body height | 1.7-2.6 | mm* |
| Tablet hardness in-process | 40-55 | N |

Coating (film coating)

| Nozzle diameter | 0.7 | mm |
|---|---|---|
| Nozzle-core bed distance | approx. 15 cm | |
| Fan jet air | 0.5 | bar |
| Atomiser air | 1 | bar |
| Internal diameter of spray hose | 3.2 | mm |
| Feed air rate | 580-600 | m³/h |
| Drum speed, heating | jog mode | |
| Drum speed, spraying | 19-20 | rpm |
| Drum speed, drying | 3-5 | rpm |
| Feed air temperature, heating | 55° C. (+/−3 K) | |
| (nominal) | | |
| Feed air temperature, spraying | 54-63° C. | |
| (actual) | | |
| Feed air temperature, drying | 65 C. (+/−3 K) | |
| (nominal) | | |
| Exhaust air temperature, beginning | 50-58° C. | |
| of spraying (actual) | | |
| Exhaust air temperature, spraying | 46-51° C. | |
| (actual) | | |
| Drying to exhaust air temperature | 59-60° C. | |
| (actual) | | |
| Spraying time | 50 | min |
| Spraying rate | 11-14 | g/min |
| Drying time | 10 | min |

*depending on ODT system and formulation

Compositions of the Ready-to-Use Mixture and the Film-Tablet Cores

a. Placebo formulation comprising Parteck® ODT (Merck):

Formulation Comprising Parteck® ODT and 1% of Parteck® LUB MST:

| | | | Proportion | Batch | SD* in |
|---|---|---|---|---|---|
| Itm. | Material No. | Material name | in % | in g | mg |
| 1 | 1.00490.9050 | Parteck ® ODT | 99 | 11,880 | 396 |
| 2 | 1.00663.9020 | Parteck ® LUB | 1 | 120 | 4 |
| | | MST (Mg. | | | |
| | | stearate) | | | |
| | | | 100 | 12,000 | 400 |

*Itm. = Item;
**SD = single-dose film-tablet core

Placebo formulation comprising Ludiflash®:

Formulation Comprising Ludiflash®+1% of Parteck® LUB MST

| | | | Proportion | Batch | SD in |
|---|---|---|---|---|---|
| Itm. | Material No. | Material name | in % | in g | mg |
| 1 | 56513304 | Ludiflash ® | 99 | 4,950 | 396 |
| | | (BASF) | | | |
| 2 | 1.00663.9020 | Parteck ® LUB | 1 | 50 | 4 |
| | | MST (Mg. | | | |
| | | stearate) | | | |
| | | | 100 | 5,000 | 400 |

Parteck® ODT (Merck) Verum Formulation

Formulation of Tablet Cores Comprising Parteck® ODT+20% of Ascorbic Acid+1% of Parteck® LUB MST

| | | | Proportion | Batch | SD in |
|---|---|---|---|---|---|
| Itm. | Material No. | Material name | in % | in g | mg |
| 1 | 1.00490.9050 | Parteck ® ODT | 79 | 5,135 | 316 |
| 2 | 83568.290 | L (+) ascorbic | 20 | 1,300 | 80 |
| | | acid | | | |
| 3 | 1.00663.9020 | Parteck ® LUB | 1 | 65 | 4 |
| | | MST (Mg. | | | |
| | | stearate) | | | |
| | | | 100 | 6,500 | 400 |

b. Ludiflash® (BASF) Verum Formulation

Formulation of Tablet Cores Comprising Ludiflash®+20% of Ascorbic Acid+1% of Parteck® LUB MST

| | | | Proportion | Batch | SD in |
|---|---|---|---|---|---|
| Itm. | Material No. | Material name | in % | in g | mg |
| 1 | 56513304 | Ludiflash ® | 79 | 3,950 | 316 |
| | | (BASF) | | | |
| 2 | 83568.290 | L (+) ascorbic | 20 | 1,000 | 80 |
| | | acid | | | |
| 3 | 1.00663.9020 | Parteck ® LUB | 1 | 50 | 4 |
| | | MST (Mg. | | | |
| | | stearate) | | | |
| | | | 100 | 5,000 | 400 |

FIG. 17 shows a comparison of the hardnesses and disintegration times of tablet cores and film tablets produced using various commercially available pre-mixes comprising different mannitol grades from various suppliers.

Composition of Coating and Film Tablets—Overview
a) Ingredients of the Colorcon and Biogrund ready-to-use coating products employed

Overview of the ingredients Opardy™ 200 White (Colorcon)

Opardy ™ 200 White (Colorcon)

Polyvinyl alcohol (PVA)
Titanium dioxide
Talc
Polyethylene glycol (macrogol)
Methacrylic acid copolymer
Sodium bicarbonate

Overview of the ingredients Aqua Polish® Clear (Biogrund)

Aqua Polish ® Clear (Biogrund)

Hydroxypropylmethylcellulose
Hydroxypropylcellulose
Talc
Modified starch
Miglyol

The amount of water was not taken into account when calculating the individual dose of the film tablet.

b) Basis Opadry 200 White (Colorcon)+Colorona Majestic Green (Merck)

Placebo formulation of film tablets for Processing Examples No. 1 and Comparative Example No. 1

| | | | Batch | Batch in | SD in |
|---|---|---|---|---|---|
| Item | Material No. | Material name | in g | g + 10% | mg |
| 1 | X | FITAB cores | 2,000 | X | 400.0 |
| | | placebo | | | |
| 2 | 200F280000 | Opadry ™ 200 | 44 | 48.40 | 8.8 |
| | | White | | | |
| 3 | 1.17190.1000 | Colorona ® | 6 | 6.60 | 1.2 |
| | | Majestic Green | | | |
| 4 | 2.00001.0000 | Aqua Purificata | 550 | 605 | 110.0 |
| | | | 2,600 | 660 | 410.0 |

c) Basis Aqua Polish@ (Biogrund)+Candurin® Brown Amber (Merck)

Placebo formulation of film tablets for Processing Example No. 2 and Comparative Example No. 2

| | | | Batch | Batch in | SD in |
|---|---|---|---|---|---|
| Item | Material No. | Material name | in g | g + 10% | mg |
| 1 | X | FITAB cores | 2,000 | X | 400.0 |
| | | placebo | | | |
| 2 | MY6128 | Aqua Polis ® Clear | 44 | 48.40 | 8.8 |
| 3 | 1.20617.1000 | Candurin ® Brown | 6 | 6.60 | 1.2 |
| | | Amber | | | |
| 4 | 2.00001.0000 | Aqua Purificata | 550 | 605 | 110.0 |
| | | | 2,600 | 660 | 410.0 |

d) Base Opadry 200 White (Colorcon)+Candurin Red Lustre (Merck)

Verum formulation of film tablets for Processing Examples No. 3 and Comparative Example No. 3

| | | | Batch | Batch in | SD |
|---|---|---|---|---|---|
| Item | Material No. | Material name | in g | g + 10% | in mg |
| 1 | X | FITAB cores | 2,000 | X | 400.0 |
| | | verum | | | |
| 2 | 200F280000 | Opadry ™ 200 | 44 | 48.40 | 8.8 |
| | | White | | | |
| 3 | 1.20619.1000 | Candurin ® Red | 6 | 6.60 | 1.2 |
| | | Lustre | | | |
| 4 | 2.00001.0000 | Aqua Purificata | 550 | 605 | 110.0 |
| | | | 2,600 | 660 | 410.0 |

e) Base Aqua Polish® (Biogrund)+Candurin® Brown Amber (Merck)

Verum formulation of film tablets for Processing Examples No. 4 and Comparative Example No. 4

| | | | Batch | Batch in | SD |
|---|---|---|---|---|---|
| Item | Material No. | Material name | in g | g + 10% | in mg |
| 1 | X | FITAB cores | 2,000 | X | 400.0 |
| | | verum | | | |
| 2 | MY6128 | Aqua Polish ® | 44 | 48.40 | 8.8 |
| | | Clear | | | |
| 3 | 1.20617.1000 | Candurin ® | 6 | 6.60 | 1.2 |
| | | Brown Amber | | | |
| 4 | 2.00001.0000 | Aqua Purificata | 550 | 605 | 110.0 |
| | | | 2,600 | 660 | 410.0 |

The amount applied was theoretically about 600 g per batch in all coating experiments, and this corresponds to about 2.4%, based on the SD. In this calculation, the amount of water was again not taken into account.

Composition and production parameters of the individual processing examples

a. Batch Size of Processing Examples No. 1 to No. 4

For the processing examples shown below, a use amount of 2000 g of film-tablet cores were in each case initially introduced into the coating drum, and an amount of coating material of 595 g was sprayed on.

b. Formulation of Processing Example No. 1

Coating of a placebo tablet (Parteck ODT) with a conventional ready-to-use coating Opadry 200 White (manufacturer Colorcon GmbH, D-65510 Idstein, Germany) plus coloured pigment Candurin Majestic Green (manufacturer Merck KGaA, D-64293 Darmstadt, Germany)

Total Overview of the Formulation for Processing Example No. 1

| Item | Material name | Proportion in % | SD in mg |
|---|---|---|---|
| 1 | Parteck ® ODT | 96.6 | 396 |
| 2 | Parteck ® LUB MST (Mg. | 1.0 | 4 |
| | stearate) | | |
| 3 | Opadry ™ 200 White | 2.1 | 8.8 |
| 4 | Colorona ® Majestic Green | 0.3 | 1.2 |
| 5 | Aqua Purificata | X | 110.0 |
| | | 100 | 410 |

From the series of experiments, the experimental results of tablets produced under identical conditions from the ready-to-use mixtures Parteck® ODT and Ludiflash® and their behaviour on coating with various coating systems are compared with one another below. These experimental results are illustrative and show the surprising advantageous behaviour of film tablets obtained on use of Parteck® ODT for the production of the tablet cores.

TABLE 1
Overview of the working examples

| | |
|---|---|
| Ex. No. | Composition |

Processing Examples

| | |
|---|---|
| No. 1 | Coating of a placebo tablet (Parteck ® ODT) with a conventional ready- |
| | to-use coating Opadry ® 200 White (manufacturer Colorcon GmbH, |
| | D-65510 Idstein, Germany) plus coloured pigment Candurin ® Majestic Green |
| | (manufacturer Merck KGaA, D-64293 Darmstadt, Germany) |
| No. 2 | Coating of a placebo tablet (Parteck ® ODT) with a conventional ready- |
| | to-use coating Aqua Polish ® Clear (manufacturer Biogrund GmbH, |
| | D-65510 Hünstetten, Germany) plus coloured pigment Candurin ® |
| | Brown Amber (manufacturer Merck KGaA, D-64293 Darmstadt, Germany) |
| No. 3 | Coating of a verum tablet comprising ascorbic acid as model active |
| | compound (Parteck ® ODT) with a conventional ready-to-use coating |
| | Opadry ® 200 White (manufacturer Colorcon GmbH, D-65510 Idstein, |
| | Germany) plus coloured pigment Candurin ® Red Lustre (manufacturer |
| | Merck KGaA, D-64293 Darmstadt, Germany) |
| No. 4 | Coating of a verum tablet comprising ascorbic acid as model active |
| | compound (Parteck ® ODT) with a conventional ready-to-use coating |
| | Aqua Polish ® Clear (manufacturer Biogrund GmbH, D-65510 |
| | Hünstetten, Germany) plus coloured pigment Candurin ® Red Lustre |
| | (manufacturer Merck KGaA, D-64293 Darmstadt, Germany) |
| No. 5 | Coating of a placebo tablet (Parteck ® ODT) with a 5% proportion of |
| | ready-to-use coating ReadiLycoat ® (manufacturer Biogrund GmbH, |
| | D-65510 Hünstetten, Germany) |
| No. 6 | Coating of a placebo tablet (Parteck ® ODT) with a 5% proportion of |
| | ready-to-use coating ReadiLycoat ® (manufacturer Biogrund GmbH, |
| | D-65510 Hünstetten, Germany) plus 4% of coloured pigment Candurin ® |
| | Silver Lustre (manufacturer Merck KGaA, D-64293 Darmstadt, Germany) |
| No. 7 | Coating of a placebo tablet (Parteck ® ODT) with a 5% proportion of |
| | ready-to-use coating ReadiLycoat ® (manufacturer Biogrund GmbH, |
| | D-65510 Hünstetten, Germany) plus 0.015% of dye Blue E133 |
| No. 8 | Coating of a placebo tablet (Parteck ® ODT) with a 5% proportion of |
| | ready-to-use coating ReadiLycoat ® (manufacturer Biogrund GmbH, |
| | D-65510 Hünstetten, Germany) plus 1% of coloured pigment Candurin ® |
| | Silver Lustre (manufacturer Merck KGaA, D-64293 Darmstadt, Germany) and |
| | 0.015% of dye Blue E133 |
| No. 9 | Coating of a placebo tablet (Parteck ® ODT) with a 5% proportion of |
| | ready-to-use coating ReadiLycoat ® (manufacturer Biogrund GmbH, |
| | D-65510 Hünstetten, Germany) plus 4% of coloured pigment Candurin ® |
| | Silver Lustre (manufacturer Merck KGaA, D-64293 Darmstadt, |
| | Germany) and 0.015% of dye Blue E133 |
| No. 10 | Analogous to Processing Example No. 5 |
| No. 11 | Coating of a verum tablet (Parteck ® ODT) with a 5% proportion of |
| | ready-to-use coating ReadiLycoat ® (manufacturer Biogrund GmbH, |
| | D-65510 Hünstetten, Germany) |

### Comparative Examples

| | |
|---|---|
| Ex. No. | Composition |

Comparative Examples

| | |
|---|---|
| No. 1 | Coating of a placebo tablet (competitor product, Ludiflash ®, BASF) with |
| | a conventional ready-to-use coating Opadry ® 200 White (manufacturer |
| | Colorcon GmbH, D-65510 Idstein, Germany) plus coloured pigment Candurin ® |
| | Majestic Green (manufacturer Merck KGaA, D-64293 Darmstadt, Germany) |
| No. 2 | Coating of a placebo tablet (competitor product, Ludiflash ®, BASF) with |
| | a conventional ready-to-use coating Aqua Polish ® Clear (manufacturer |
| | Biogrund GmbH, D-65510 Hünstetten, Germany) plus coloured pigment |
| | Candurin ® Brown Amber (manufacturer Merck KGaA, D-64293 |
| | Darmstadt, Germany) |
| No. 3 | Coating of a verum tablet comprising ascorbic acid as model active |
| | compound (competitor product, Ludiflash ®, BASF) with a conventional |
| | ready-to-use coating Opadry ® 200 White (manufacturer Colorcon |
| | GmbH, D-65510 Idstein, Germany) plus coloured pigment Candurin ® |
| | Red Lustre (manufacturer Merck KGaA, D-64293 Darmstadt, Germany) |
| No. 4 | Coating of a verum tablet comprising ascorbic acid as model active |
| | compound (competitor product, Ludiflash ®, BASF) with a conventional |
| | ready-to-use coating Aqua Polish ® Clear (manufacturer Biogrund |
| | GmbH, D-65510 Hünstetten, Germany) plus coloured pigment Candurin ® |
| | Red Lustre (manufacturer Merck KGaA, D-64293 Darmstadt, Germany) |
| No. 5 | Coating of a placebo tablet (competitor product, Ludiflash ®, BASF) with |
| | a 5% proportion of ready-to-use coating ReadiLycoat ® (manufacturer |
| | Biogrund GmbH, D-65510 Hünstetten, Germany) |
| No. 6 | Coating of a verum tablet (competitor product, Ludiflash ®, BASF) with a |
| | 5% proportion of ready-to-use coating ReadiLycoat ® (manufacturer |
| | Biogrund GmbH, D-65510 Hünstetten, Germany) |

Comparison of the data for Parteck®ODT (Merck)/Ludiflash® (BASF)—water-based coating

Key to the Abbreviations:

FITAB=film tablet

TABLTK=tablet core (film-tablet core)

Comparison of the Placebo Recipes—Coating with Opadry® 200:

Processing Examples No. 1 and Comparative Example No. 1

### Processing Example No. 1

Coating of a placebo tablet (Parteck® ODT) with a conventional ready-to-use coating Opadry® 200 White (manufacturer Colorcon GmbH, D-65510 Idstein, Germany) plus coloured pigment Candurin® Majestic Green (manufacturer Merck KGaA, D-64293 Darmstadt, Germany)

### Comparative Example No. 1

Coating of a placebo tablet (Ludiflash®, BASF) with a conventional ready-to-use coating Opadry® 200 White (manufacturer Colorcon GmbH, D-65510 Idstein, Germany) plus coloured pigment Candurin® Majestic Green (manufacturer Merck KGaA, D-64293 Darmstadt, Germany)

TABLE 2
Comparison of Processing Example No. 1 and Comparative
Example No. 1

| | | Comparative |
|---|---|---|
| | Processing Example | Example No. 1 |
| | No. 1 | Ludiflash ® + |
| | Parteck ® ODT + | 1% of Parteck ® |
| | 1% of Parteck ® LUB | LUB MST + |
| | MST + Opadry ™ 200 + | Opadry ™ 200 + |
| | Colorona ® Majestic | Colorona ® |
| Parameter | Green | MajesticGreen |
| TABLTK hardness after 1 | 51 | 44 |
| day [N] | | |
| s.rel in % | 7.89 | 7.31 |
| FITAB hardness after 1 day | 108 (+112%) | 93 (+111%) |
| [N] | | |
| s.rel in % | 11.58 | 10.45 |
| Tablet weight [mg] | 399.95 | 407.83 |
| s.rel in % | 0.65 | 0.55 |
| Film-tablet weight [mg] | 405.40 | 412.90 |
| s.rel in % | 2.37 | 11.16 |
| Amount of coating applied | 5.45 | 5.07 |
| [mg] | | |
| Disintegration time, 37° C. | 56 | 40 |
| TABLTK [sec] | | |
| Disintegration time, 37° C. | 53 (−5%) | 501 (+1153%) |
| FITAB [sec] | | |
| Friability TABLTK [%] | 0.37 | 0.36 |
| Friability FITAB [%] | 0.00 | 0.00 |

### 1.1.1. Comparison of Appearance of the Film Tablets from Processing Example No. 1 with Comparative Example No. 1

### 1.1.2. Comparison of the Properties Hardness and Disintegration Time of Processing Example No. 1 with Comparative Example No. 1

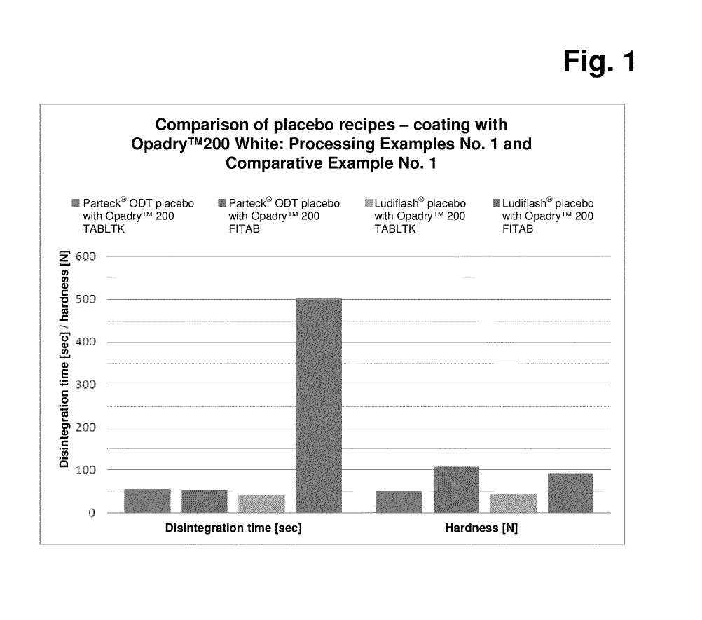

FIG. 1: Depiction of the evaluation of the comparison of the hardnesses and disintegration times before and after production of the coating of Processing Example No. 1 and Comparative Example No. 1

The Parteck® ODT placebo tablet cores have a disintegration time of 56 seconds with a hardness of 51 N, and similar properties are also exhibited by the Ludiflash® placebo tablet cores, with a disintegration time of 40 seconds with a tablet hardness of 44 N.

After coating with Opadry™ 200 White, however, considerable differences are observed. Thus, the hardness of the Parteck® ODT placebo film tablets increases to 108 N and the disintegration time is nevertheless only 53 seconds. By contrast, the disintegration time of the Ludiflash® placebo film tablets increases to 501 seconds with a hardness of 93 N.

### 1.2. Comparison of the Placebo Recipes—Coating with Aqua Polish® Clear: Processing Examples No. 2 and Comparative Example No. 2

### Processing Example No. 2

Coating of a placebo tablet (Parteck ODT) with a conventional ready-to-use coating Aqua Polish Clear (manufacturer Biogrund GmbH, D-65510 Hünstetten, Germany) plus coloured pigment Candurin Brown Amber (manufacturer Merck KGaA, D-64293 Darmstadt, Germany)

### Comparative Example No. 2

Coating of a placebo tablet (competitor product, Ludiflash, BASF) with a conventional ready-to-use coating Aqua Polish Clear (manufacturer Biogrund GmbH, D-65510 Hünstetten, Germany) plus coloured pigment Candurin Brown Amber (manufacturer Merck KGaA, D-64293 Darmstadt, Germany)

TABLE 3
Comparison of Processing Examples No. 2 and Comparative
Example No. 2

| | Parteck ® ODT + | Ludiflash ® +1% of |
|---|---|---|
| | 1% of Parteck ® | Parteck ® |
| | LUB MST + Aqua | LUB MST + Aqua |
| | Polish ® Clear + | Polish ® Clear + |
| | Candurin ® | Candurin ® |
| Parameter | Brown Amber | Brown Amber |
| TABLTK hardness after 1 | 51 | 44 |
| day [N] | | |
| s.rel in % | 7.89 | 7.31 |
| FITAB hardness after 1 day | 90 (+77%) | 93 (+111%) |
| [N] | | |
| s.rel in % | 9.09 | 8.05 |
| Tablet weight [mg] | 399.95 | 407.83 |
| s.rel in % | 0.65 | 0.55 |
| Film-tablet weight [mg] | 407.50 | 416.40 |
| s.rel in % | 3.30 | 1.68 |
| Amount of coating applied | 7.55 | 8.75 |
| [mg] | | |
| Disintegration time, 37° C. | 56 | 40 |
| TABLTK [sec] | | |
| Disintegration time, 37° C. | 79 (+41%) | 400 (+900%) |
| FITAB [sec] | | |
| Friability TABLTK [%] | 0.37 | 0.36 |
| Friability FITAB [%] | 0.00 | 0.00 |

Comparison of Appearance of the Film Tablets from Processing Example No. 2 with Comparative Example No. 2

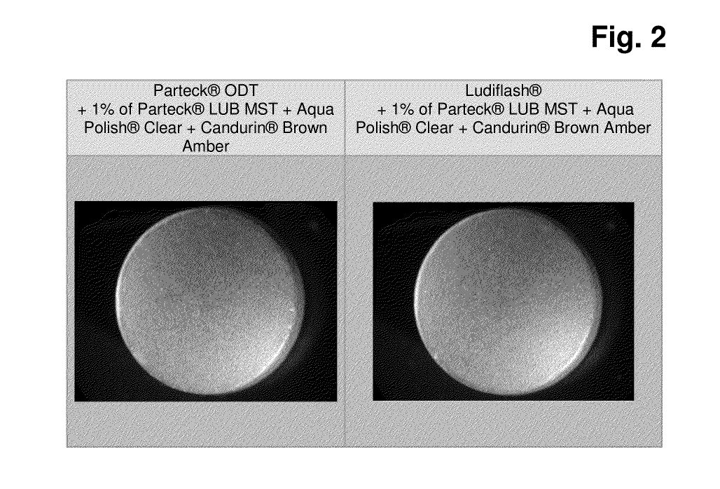

FIG. 2: Comparison of appearance of FITAB Parteck® ODT placebo and Ludiflash® placebo coated with Aqua Polish® Clear

### 1.2.1. Conclusion of the Comparison of Processing Example No. 2 with Comparative Example No. 2

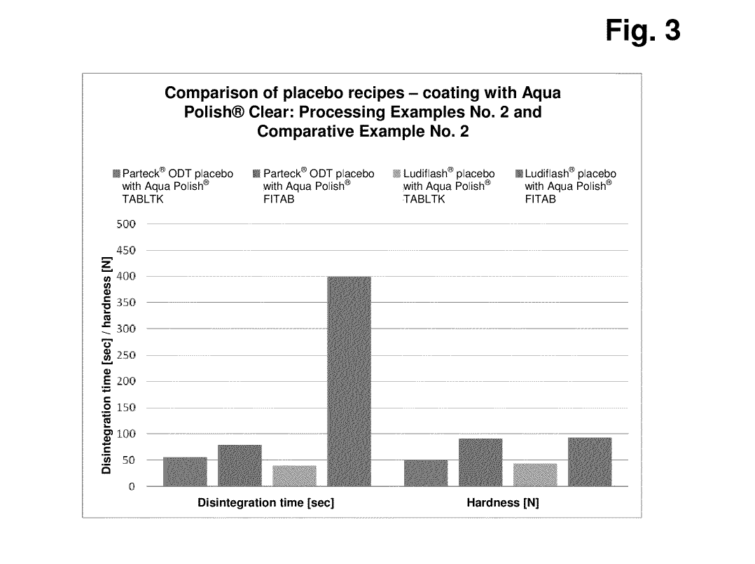

FIG. 3: Depiction of the evaluation of the comparison of Processing Example No. 2 and Comparative Example No. 2

The Parteck® ODT placebo tablet cores have a disintegration time of 56 seconds with a hardness of 51 N, and similar properties are also exhibited by the Ludiflash® placebo tablet cores, with a disintegration time of 40 seconds and a tablet hardness of 44 N.

After coating with Opadry™ 200 White, however, significant differences are observed. Thus, the hardness of the Parteck® ODT placebo film tablets increases to 90 N (+77%) and the disintegration time is only 79 seconds (+41%). By contrast, the disintegration time of the Ludiflash® placebo film tablets increases to 400 seconds (+900%) with a hardness of 93 N (+111%).

### 1.3. Comparison of the Active-Compound-Containing Recipes—Coating with Opadry™ 200: Processing Examples No. 3 and Comparative Example No. 3

### Processing Example No. 3

Coating of a verum tablet with ascorbic acid as model active compound (Parteck ODT) with a conventional ready-to-use coating Opadry 200 White (manufacturer Colorcon GmbH, D-65510 Idstein, Germany) plus coloured pigment Candurin Red Lustre (manufacturer Merck KGaA, D-64293 Darmstadt, Germany)

### Comparative Example No. 3

Coating of a verum tablet with ascorbic acid as model active compound (competitor product, Ludiflash, BASF) with a conventional ready-to-use coating Opadry 200 White (manufacturer Colorcon GmbH, D-65510 Idstein, Germany) plus coloured pigment Candurin Red Lustre (manufacturer Merck KGaA, D-64293 Darmstadt, Germany)

TABLE 4
Comparison of Processing Examples No. 3 and
Comparative Example No. 3

| | | Ludiflash ® + |
|---|---|---|
| | | 20% of |
| | | ascorbic acid + |
| | Parteck ® ODT + | 1% of Parteck ® |
| | 20% of ascorbic acid + | LUB MST + |
| | 1% of Parteck ® LUB | Opadry ™ 200 + |
| | MST + Opadry ™ 200 + | Candurin ® |
| Parameter | Candurin ® Red Lustre | Red Lustre |
| TABLTK hardness after 1 | 54 | 45 |
| day [N] | | |
| s.rel in % | 7.14 | 10.24 |
| FITAB hardness after 1 day | 117 (+117%) | 62 (+38%) |
| [N] | | |
| s.rel in % | 10.55 | 10.26 |
| Tablet weight [mg] | 408.60 | 403.58 |
| s.rel in % | 0.58 | 0.60 |
| Film-tablet weight [mg] | 417.10 | 408.00 |
| s.rel in % | 3.55 | 3.17 |
| Amount of coating applied | 8.50 | 4.42 |
| [mg] | | |
| Disintegration time, 37° C. | 28 | 22 |
| TABLTK [sec] | | |
| Disintegration time, 37° C. | 55 (+96%) | 309 (+1305%) |
| FITAB [sec] | | |
| Friability TABLTK [%] | 0.45 | 14.71 |
| Friability FITAB [%] | 0.00 | 0.00 |

### 1.3.1. Comparison of Appearance of the Film Tablets of Processing Example No. 3 with Comparative Example No. 3

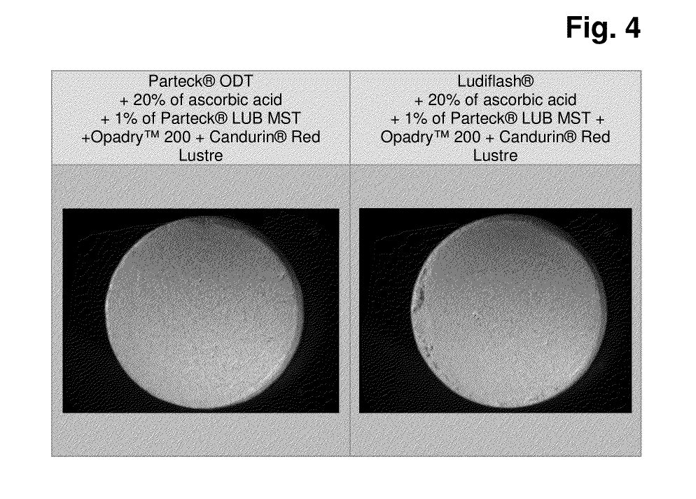

FIG. 4: Comparison of the appearance of FITAB Parteck® ODT verum and Ludiflash® verum coated with Opadry™ 200

While the film tablet produced from Parteck® ODT has a relatively uniform edge, the film tablet produced from Ludiflash exhibits considerable fracturing at the edge.

### 1.3.2. Conclusion of the Comparison of Processing Example No. 2 with Comparative Example No. 2

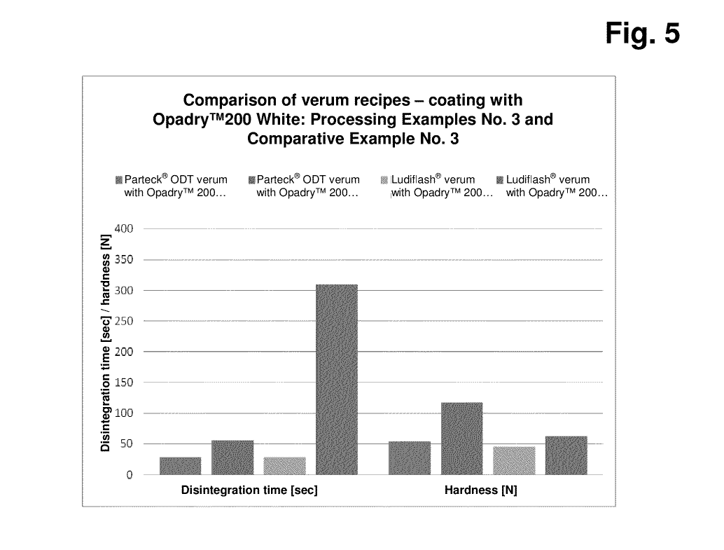

FIG. 5: Depiction of the evaluation of the comparison of Processing Example No. 3 and Comparative Example No. 3

The Parteck® ODT placebo tablet cores have a disintegration time of 28 seconds with a hardness of 54 N, and similar properties are also exhibited by the Ludiflash® placebo tablet cores, with a disintegration time of 22 seconds with a tablet hardness of 45 N.

After coating with Opadry™ 200 White, however, significant differences are observed. Thus, the hardness of the Parteck® ODT placebo film tablets increases to 117 N (+117%) and the disintegration time is nevertheless only 55 seconds (+96%). By contrast, the disintegration time of the Ludiflash® placebo film tablets increases to 309 seconds (+1305%) with a hardness of 62 N (+38%).

### 1.4. Comparison of the Verum Recipes—Coating with Aqua Polish® Clear: Processing Examples No. 4 and Comparative Example No. 4

### Processing Example No. 4

Coating of a verum tablet with ascorbic acid as model active compound (Parteck ODT) with a conventional ready-to-use coating Aqua Polish Clear (manufacturer Biogrund GmbH, D-65510 Hünstetten, Germany) plus coloured pigment Candurin Red Lustre (manufacturer Merck KGaA, D-64293 Darmstadt, Germany)

### Comparative Example No. 4

Coating of a verum tablet with ascorbic acid as model active compound (competitor product, Ludiflash) with a conventional ready-to-use coating Aqua Polish Clear (manufacturer Biogrund GmbH, D-65510 Hünstetten, Germany) plus coloured pigment Candurin Red Lustre (manufacturer Merck KGaA, D-64293 Darmstadt, Germany)

TABLE 5
Comparison of Processing Examples No. 4 and Comparative
Example No. 4

| | | Ludiflash ® + |
|---|---|---|
| | | 20% of ascorbic |
| | Parteck ® ODT + | acid + 1% of |
| | 20% of ascorbic acid + | Parteck ® LUB |
| | 1% of Parteck ® LUB | MST + Aqua |
| | MST + Aqua Polish ® | Polish ® Clear + |
| | Clear + Candurin ® Red | Candurin ® Red |
| Parameter | Lustre | Lustre |
| TABLTK hardness after 1 | 54 | 45 |
| day [N] | | |
| s.rel in % | 7.14 | 10.24 |
| FITAB hardness after 1 | 93 (+72%) | 59 (+31%) |
| day [N] | | |
| s.rel in % | 4.70 | 10.88 |
| Tablet weight [mg] | 408.60 | 403.58 |
| s.rel in % | 0.58 | 0.60 |
| Film-tablet weight [mg] | 417.3 | 403.00 |
| s.rel in % | 2.56 | 8.41 |
| Amount of coating applied | 8.70 | n.d.* |
| [mg] | | |
| Disintegration time, 37° C. | 28 | 22 |
| TABLTK [sec] | | |
| Disintegration time, 37° C. | 74 (+164%) | 175 (+696%) |
| FITAB [sec] | | |
| Friability TABLTK [%] | 0.45 | 14.71 |
| Friability FITAB [%] | 0.00 | 0.00 |

*The amount of coating applied could not be determined since the tablet cores crumbled during coating owing to the mechanical load and their only low stability

### 1.4.1. Comparison of Appearance of the Film Tablets of Processing Example No. 4 with Comparative Example No. 4

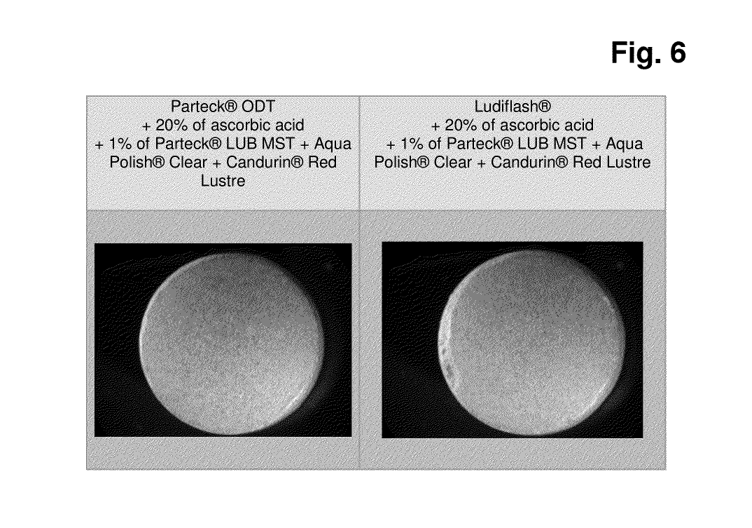

FIG. 6: Comparison of appearance of FITAB Parteck® ODT verum and Ludiflash® verum coated with Aqua Polish® Clear

While the film tablet produced from Parteck® ODT has a relatively uniform edge, the film tablet produced from Ludiflash exhibits considerable fracturing at the edge.

### 1.4.2. Conclusion of the Comparison of Processing Example No. 2 with Comparative Example No. 2

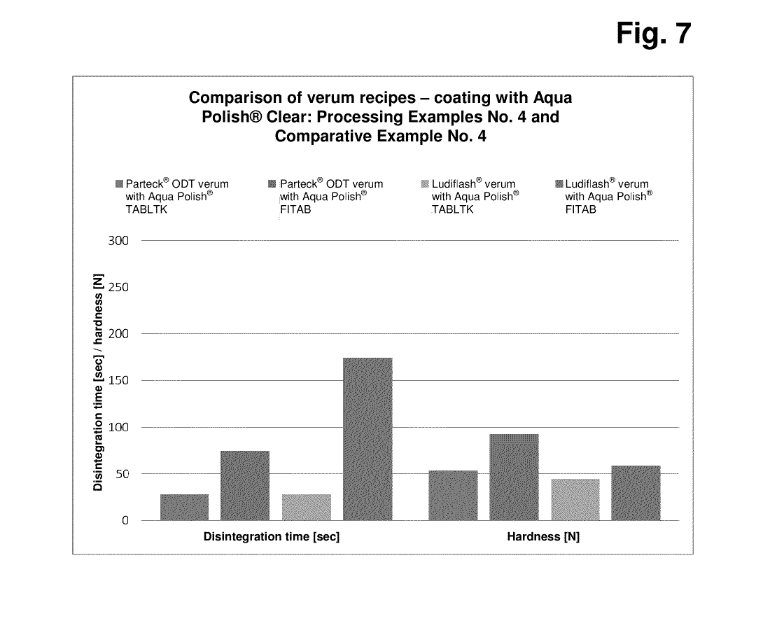

FIG. 7: Depiction of the evaluation of the comparison of Processing Example No. 4 and Comparative Example No. 4

The Parteck® ODT placebo tablet cores have a disintegration time of 28 seconds with a hardness of 54 N, and similar properties are also exhibited by the Ludiflash® placebo tablet cores, with a disintegration time of 22 seconds with a tablet hardness of 45 N.

After coating with Opadry™ 200 White, however, significant differences are observed. Thus, the hardness of the Parteck® ODT placebo film tablets increases to 93 N (+72%) and the disintegration time is nevertheless only 74 seconds (+164%). By contrast, the disintegration time of the Ludiflash® placebo film tablets increases to 175 seconds (+696%) with a hardness of 59 N (+31%).

### 1.5. Effect of the Coating on the Disintegration Time and Hardness of the Processing and Comparative Examples

### 1.5.1. Effects of the Coating on the Disintegration Time and Hardness of The Placebo Processing and Comparative Examples

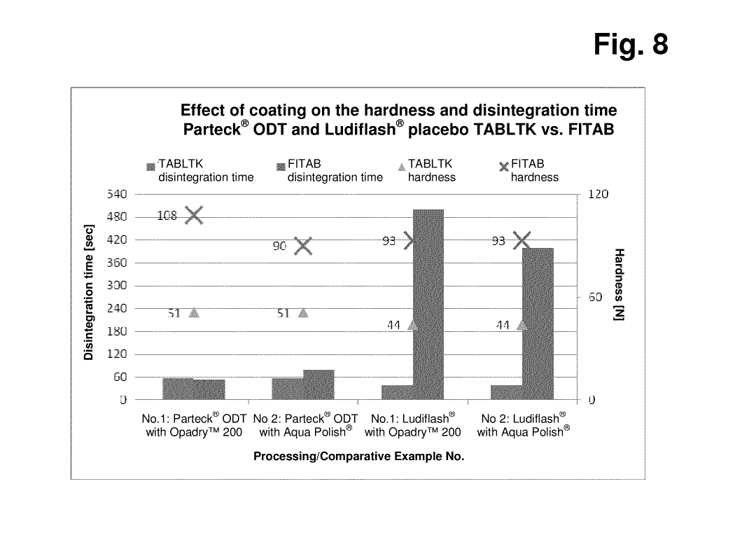

FIG. 8: Effects of the placebo formulations in comparison

While the disintegration times only change insignificantly with application of the coating film to the Parteck® ODT tablets, the disintegration time increases to more than 300 seconds in the case of Ludiflash® tablets, meaning that it is no longer a rapidly disintegrating tablet.

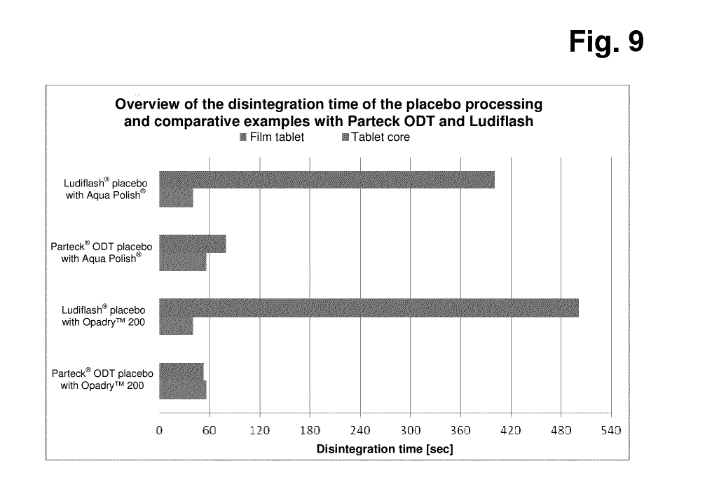

FIG. 9: Consideration of the disintegration time of the placebo formulations in comparison

The same applies here as in the case of FIG. 9

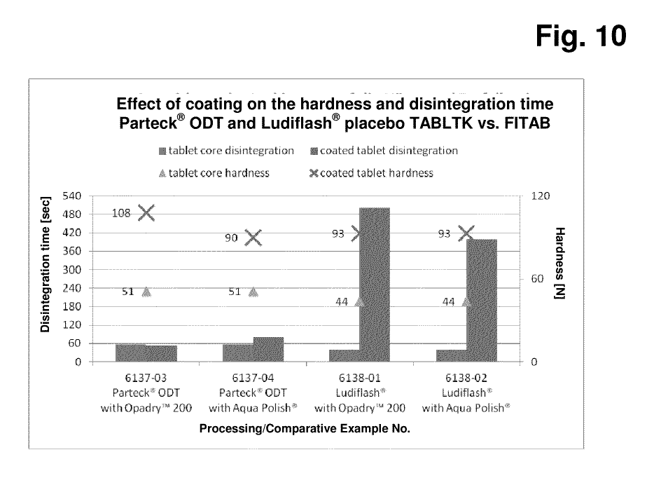

FIG. 10: Effect of the verum formulations in comparison

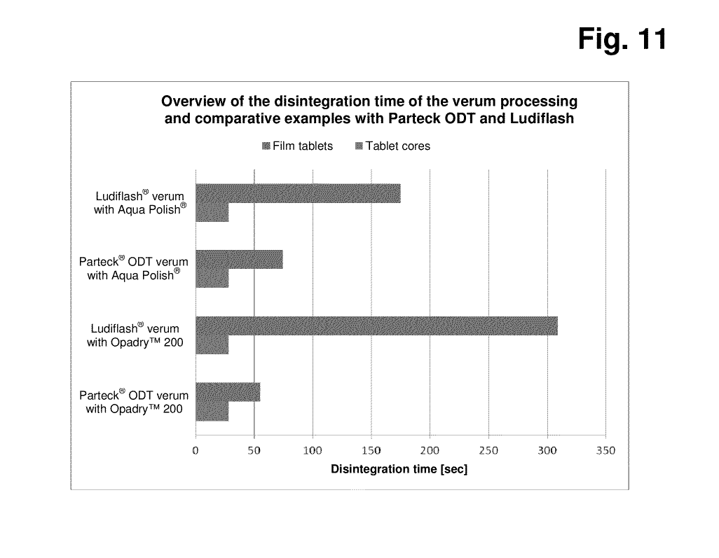

FIG. 11: Consideration of the disintegration time of the verum formulations in comparison

### 2. Comparison of the Coating Experiments with Tablet Cores Produced From the Ready-to-Use Mixtures Parteck®ODT (Merck) And Ludiflash® (BASF) with Coating Compositions Based on Water/Ethanol

### 2.1. Comparison of the Placebo Recipes—Coating with ReadiLycoat D Clear 590.03 G: Processing Examples No. 10 and Comparative Example No. 5

### Processing Example No. 10

Coating of a placebo tablet (Parteck ODT) with a 5% proportion of ready-to-use coating ReadiLycoat (manufacturer Biogrund GmbH, D-65510 Hünstetten, Germany)

### Comparative Example No. 5

Coating of a placebo tablet (competitor product, Ludiflash, BASF) with a 5% proportion of ready-to-use coating ReadiLycoat (manufacturer Biogrund GmbH, D-65510 Hünstetten, Germany)

TABLE 6
Comparison of Processing Examples No. 10 with Comparative
Example No. 5

| | Parteck ® ODT + | Ludiflash ® + |
|---|---|---|
| | 1% of Parteck ® LUB | 1% of Parteck ® |
| | MST + 5% of | LUB MST + 5% |
| Parameter | ReadiLycoat | of ReadiLycoat |
| TABLTK hardness after 1 day | 51 | 44 |
| [N] | | |
| s.rel in % | 7.89 | 7.31 |
| FITAB hardness after 1 day | 91 (+78%) | 72 (+64%) |
| [N] | | |
| s.rel in % | 6.76 | 9.94 |
| Tablet weight [mg] | 399.95 | 407.83 |
| s.rel in % | 0.65 | 0.55 |
| Film-tablet weight [mg] | 399.70 | 408.10 |
| s.rel in % | 0.50 | 0.48 |
| Amount of coating applied | −0.25 | +0.27 |
| [mg] | | |
| Disintegration time, 37° C. | 56 | 40 |
| TABLTK [sec] | | |
| Disintegration time, 37° C. | 49 (−13%) | 61 (+53%) |
| FITAB [sec] | | |
| Friability TABLTK [%] | 0.37 | 0.36 |
| Friability FITAB [%] | 0.21 | 0.23 |

Comparison of Outer Appearance of the Film Tablets of Processing Example No. 10 and Comparative Example No. 5

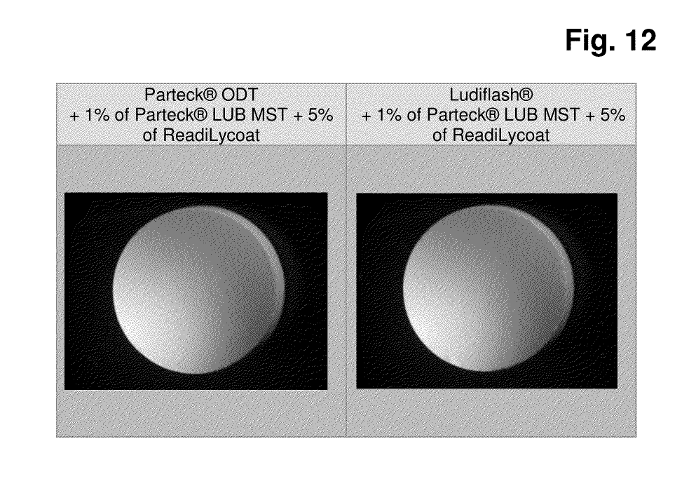

FIG. 12: Comparison of the appearance of FITAB Parteck® ODT placebo and Ludiflash® placebo coated with ReadiLycoat

### 2.1.1. Conclusion of the Comparison of Processing Example No. 10 with Comparative Example No. 5

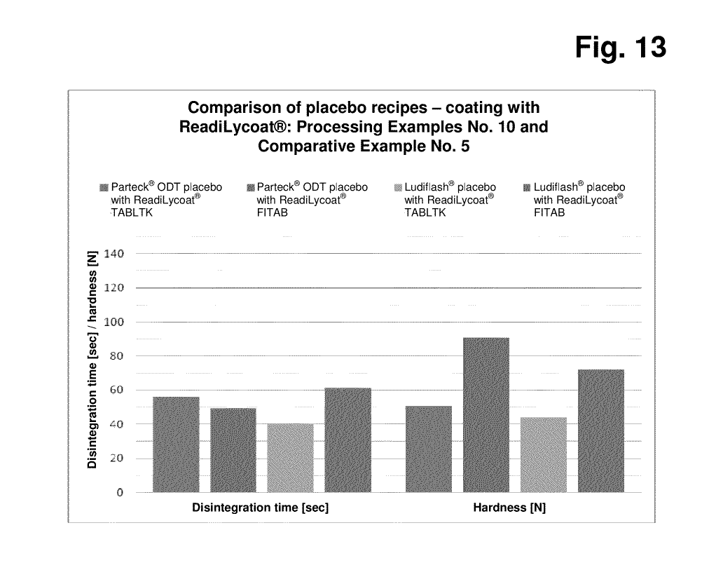

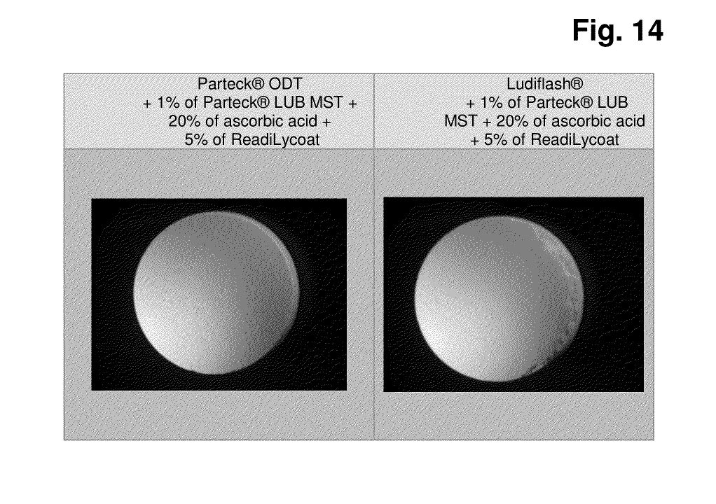

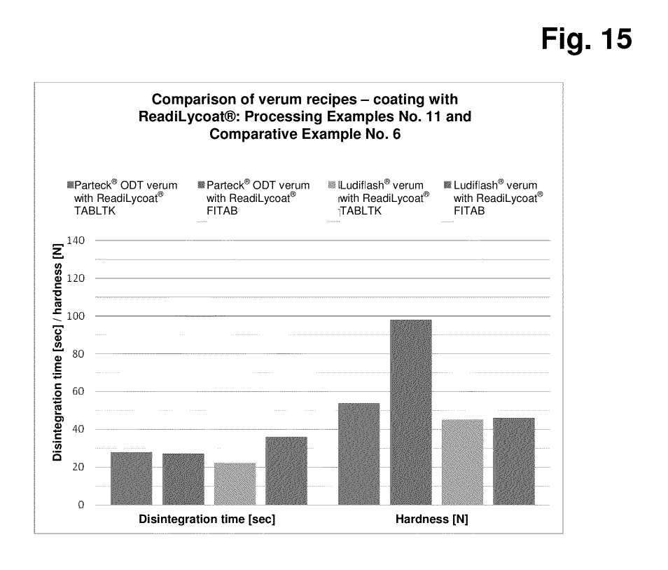

FIG. 13: Depiction of the evaluation of the comparison of Processing Example No. 10 and Comparative Example No. 5

### 2.2. Comparison of Active-Compound-Containing Tablets—Coating with ReadiLycoat D Clear 590.03 G: Processing Examples No. 11 and Comparative Example No. 6

### Processing Example No. 11

Coating of a verum tablet (Parteck ODT) with a 5% proportion of ready-to-use coating ReadiLycoat (manufacturer Biogrund GmbH, D-65510 Hünstetten, Germany)

### Comparative Example No. 6

Coating of a verum tablet (competitor product, Ludiflash, BASF) with a 5% proportion of ready-to-use coating ReadiLycoat (manufacturer Biogrund GmbH, D-65510 Hünstetten, Germany)

TABLE 7
Comparison of Processing Examples No. 11 and
Comparative Example No. 6

| | | Ludiflash ® + |
|---|---|---|
| | Parteck ® ODT + | 1% of Parteck ® |
| | 1% of Parteck ® LUB | LUB MST + |
| | MST + 20% of ascorbic | 20% of ascorbic |
| | acid + | acid + 5% |
| Parameter | 5% of ReadiLycoat | of ReadiLycoat |
| TABLTK hardness after | 54 | 45 |
| 1 day [N] | | |
| s.rel in % | 7.14 | 10.24 |
| FITAB hardness after 1 | 98 (+82%) | 46 (+2%) |
| day [N] | | |
| s.rel in % | 7.06 | 14.36 |
| Tablet weight [mg] | 408.60 | 403.58 |
| s.rel in % | 0.58 | 0.60 |
| Film-tablet weight [mg] | 409.30 | 402.40 |
| s.rel in % | 0.84 | 0.48 |
| Amount of coating | +0.70 | n.d.* |
| applied [mg] | | |
| Disintegration time, 37° C. | 28 | 22 |
| TABLTK [sec] | | |
| Disintegration time, | 27 (−4%) | 36 (+64%) |
| 37° C. FITAB [sec] | | |
| Friability TABLTK [%] | 0.45 | 14.71 |
| Friability FITAB [%] | 0.35 | 6.33 |

*The amount of coating applied could not be determined since the tablet cores crumbled during coating owing to the mechanical load and their only low stability.

### 2.2.2. Comparison of Appearance of the Film Tablets of Processing Example No. 11 and Comparative Example No. 6

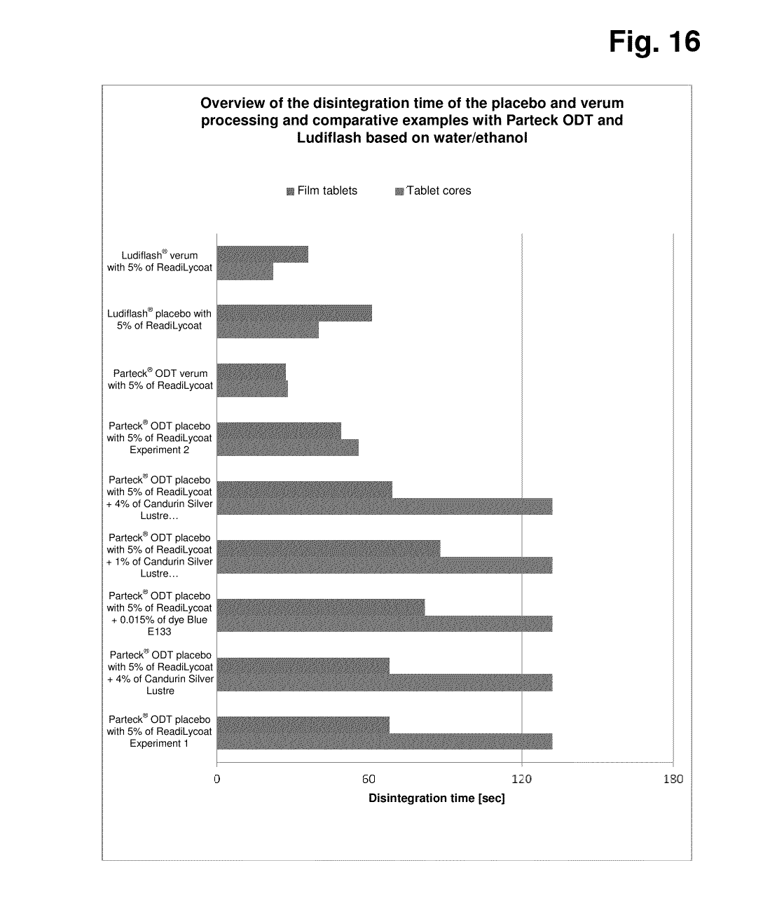

FIG. 14: Comparison of appearance of FITAB Parteck® ODT verum and Ludiflash® verum coated with ReadiLycoat

While the ascorbic acid-containing Parteck® ODT film tablet has only extremely small irregularities at the edge, the corresponding Ludiflash® tablet exhibits extremely irregular edges and significant irregularities on the surface.

Pharmaceutical formulation properties of the tablets or film
tablets of the ODT systems employed

| | Parteck ® ODT | Ludiflash ® | Pearlitol ® Flash |
|---|---|---|---|
| | (Merck) | (BASF) | (Roquette) |
| | +1% of Parteck ® LUB | +1% of Parteck ® LUB | +1% of Parteck ® LUB |
| | MST | MST | MST |
| | +2.15% of Opadry ™ | +2.15% of Opadry ™ | +2.15% of Opadry ™ |
| | 200 | 200 | 200 |
| | +0.3% of Colorona ® | +0.3% of Colorona ® | +0.3% of Colorona ® |
| Parameter | Majectic Green | Majectic Green | Majectic Green |
| TABLTK | 50 | 50 | 54 |
| hardness in- | | | |
| process [N] | | | |
| (nominal = 50 | | | |
| N) | | | |
| Relative | 7.93 | 7.90 | 6.67 |
| standard | | | |
| deviation [%] | | | |
| TABLTK | 51 | 44 | 46 |
| hardness after | | | |
| 1 day [N] | | | |
| Relative | 7.89 | 7.31 | 8.36 |
| standard | | | |
| deviation [%] | | | |
| FITAB | 108 (+112%) | 93 (+111%) | 106 (+130%) |
| hardness after | | | |
| 1 day [N] | | | |
| Relative | 11.58 | 10.45 | 8.36 |
| standard | | | |
| deviation [%] | | | |
| | Prosolv ® ODT | F-Melt ® Type C | F-Melt ® Type M |
| | (JRS Pharma) | (Fuji Chemical) | (Fuji Chemical) |
| | +1% of Parteck ® LUB | +1% of Parteck ® LUB | +1% of Parteck ® LUB |
| | MST | MST | MST |
| | +2.15% of Opadry ™ | +2.15% of Opadry ™ | +2.15% of Opadry ™ |
| | 200 | 200 | 200 |
| | +0.3% of Colorona ® | +0.3% of Colorona ® | +0.3% of Colorona ® |
| Parameter | Majectic Green | Majectic Green | Majectic Green |
| TABLTK | 51 | 50 | 55 |
| hardness in- | | | |
| process [N] | | | |
| (nominal = 50 | | | |
| N) | | | |
| Relative | 7.65 | 7.42 | 7.59 |
| standard | | | |
| deviation [%] | | | |
| TABLTK | 44 | 45 | 53 |
| hardness after | | | |
| 1 day [N] | | | |
| Relative | 11.05 | 8.90 | 7.45 |
| standard | | | |
| deviation [%] | | | |
| FITAB | 112 (+155%) | 113 (+151%) | 139 (+162%) |
| hardness after | | | |
| 1 day [N] | | | |
| Relative | 11.05 | 8.90 | 7.45 |
| standard | | | |
| deviation [%] | | | |
| | Parteck ® ODT | Ludiflash ® | Pearlitol ® Flash |
| | (Merck) | (BASF) | (Roquette) |
| | +1% of Parteck ® LUB | +1% of Parteck ® LUB | +1% of Parteck ® LUB |
| | MST | MST | MST |
| | +2.15% of Opadry ™ | +2.15% of Opadry ™ | +2.15% of Opadry ™ |
| | 200 | 200 | 200 |
| | +0.3% of Colorona ® | +0.3% of Colorona ® | +0.3% of Colorona ® |
| Parameter | Majectic Green | Majectic Green | Majectic Green |
| TABLTK mass | 400.0 | 407.8 | 401.0 |
| [mg] (nominal = | | | |
| 400 mg) | | | |
| Relative | 0.65 | 0.55 | 0.48 |
| standard | | | |
| deviation [%] | | | |
| Disintegration | 56 | 40 | 54 |
| time, 37° C. | | | |
| TABKTK [sec] | | | |
| Disintegration | 53 (−5%) | 501 (+1153%) | 80 (+48%) |
| time, 37° C. | | | |
| FITAB [sec] | | | |
| Fribility | 0.37 | 0.36 | 0.33 |
| TABLTK [%] | | | |
| Fribility | 0.00 | 0.00 | 0.00 |
| FITAB [%] | | | |
| Appearance | smooth, slight damage | smooth, slight damage | smooth, some abrasion |
| FITAB | at the edge, mint- | at the edge, mint- | at the edge, mint- |
| | green/white-speckled | green/white-speckled | green/white-speckled |
| | glossy | glossy | glossy |
| | Prosolv ® ODT | F-Melt ® Type C | F-Melt ® Type M |
| | (JRS Pharma) | (Fuji Chemical) | (Fuji Chemical) |
| | +1% of Parteck ® LUB | +1% of Parteck ® LUB | +1% of Parteck ® LUB |
| | MST | MST | MST |
| | +2.15% of Opadry ™ | +2.15% of Opadry ™ | +2.15% of Opadry ™ |
| | 200 | 200 | 200 |
| | +0.3% of Colorona ® | +0.3% of Colorona ® | +0.3% of Colorona ® |
| Parameter | Majectic Green | Majectic Green | Majectic Green |
| TABLTK mass | 402.4 | 407.5 | 404.0 |
| [mg] (nominal = | | | |
| 400 mg) | | | |
| Relative | 0.64 | 0.74 | 0.52 |
| standard | | | |
| deviation [%] | | | |
| Disintegration | 115 | 17 | 66 |
| time, 37° C. | | | |
| TABKTK [sec] | | | |
| Disintegration | 201 (+75%) | 79 (+365%) | 138 (+109%) |
| time, 37° C. | | | |
| FITAB [sec] | | | |
| Fribility | 0.67 | 0.30 | 1.34 |
| TABLTK [%] | | | |
| Fribility | | | |
| FITAB [%] | 0.00 | 0.00 | 0.00 |
| Appearance | somewhat rough, mint- | smooth, mint- | smooth, mint- |
| FITAB | green/white-speckled | green/white-speckled | green/white-speckled |
| | slightly glossy | slightly glossy | slightly glossy |

TABLTK = tablet core (tablet core before coating),
FITAB = film tablet (tablet core after coating → coated film tablet)
Considerable or significant differences from Parteck ® ODT are highlighted in bold

1. Pharmaceutical formulation in the form of a coated tablet, characterised in that it consists of
a) a tablet core, obtainable from a homogenised, directly compressible co-mixture of spray-granulated mannitol and crosslinked croscarmellose-sodium and at least one pharmaceutical active compound or food supplement and additives, and
b) a coating which is applied in the form of an aqueous or water- and alcohol-containing solution,
and in that it is a tablet which disintegrates rapidly in the presence of moisture.

2. Pharmaceutical formulation according to claim 1, characterised in that the co-mixture used for the production of the tablet core comprises 90 to 95% by weight of mannitol and 3 to 7% by weight of croscarmellose-sodium as tablet disintegrant, and optionally up to 1% by weight of magnesium stearate.

3. Pharmaceutical formulation according to claim 1, characterised in that the co-mixture used for the production of the tablet core has a flow angle in the range from 33 to 38°, particle sizes in the range from 70 to 120 μm (D₅₀; laser), a bulk density in the range from 0.55 to 0.65 g/ml and a tapped density in the range from 0.70 to 0.80 g/ml.

4. Pharmaceutical formulation according to claim 1, characterised in that the co-mixture used for the production of the tablet core has a BET surface area in the range from 2.4 to 3.5 m²/g.

5. Pharmaceutical formulation according to claim 1, characterised in that the tablet core comprises a pharmaceutical active compound or food supplement in an amount of 0.1 to 50% by weight, based the weight of the tablet core.

6. Pharmaceutical formulation according to claim 1, characterised in that the tablet core comprises glidants or lubricants in the form of magnesium stearate, sodium stearyl fumarate, stearic acid, polyethylene glycol (PEG 6000) in an amount of up to 0.1 to 5% by weight, based the weight of the tablet core.

7. Pharmaceutical formulation according to claim 1, characterised in that the coating is applied in the form of a water- or water/ethanol-containing solution.

8. Pharmaceutical formulation according to claim 1, characterised in that the coating comprises soluble film formers from the group polyvinylpyrrolidone, vinylpyrrolidone-vinyl acetate copolymer, polyvinyl acetate, hydroxypropylmethylcellulose, methacrylate copolymer or mixtures thereof.

9. Pharmaceutical formulation according to claim 1, characterised in that the coating is produced from a solution which, besides one or more film formers, comprise one or more sugars from the group glucose, dextrose, fructose, lactose, maltose, xylose, sucrose, corn syrup, sorbitol, hexitol, maltitol, xylitol and mannitol, optionally at least one polyalcohol selected from the group glycerol, polyethylene glycol and propylene glycol, and optionally at least one edible acid which is suitable for foods, from the group citric acid, malic acid, tartaric acid, fumaric acid, phosphoric acid, oxalic acid and ascorbic acid, and aroma oils and/or flavours, which have a pleasant effect in the mouth even during dissolution of the outer tablet coating.

10. Pharmaceutical formulation according to claim 1, characterised in that the tablet core comprises an active compound selected from the group atypical antipsychotics, antipsychotics, antidepressants, antihistamines, acetylcholinesterase inhibitors, analgesics, antipyretics, anticonvulsant, anticholinergic, antiemetics, benzodiazepines, corticosteroids, DDC inhibitors [carbidopa], dopamine receptor antagonists, monoamine oxidase inhibitors (MAOIs), non-benzodiazepine hypnotics, opioid analgesic [tramadol], proton pump inhibitors, triptans/serotonin agonists, NSAIDs and SSRIs.

11. Pharmaceutical formulation according to claim 1, characterised in that the tablet core has low abrasion of less than 0.50%, based on the weight.

12. Process for the preparation of a pharmaceutical formulation according to claim 1, characterised in that the tablet cores produced are warmed to an elevated temperature in a coating drum with mixing, and the coating is produced by spraying the low-viscosity coating solution onto the tablet cores and drying at elevated temperature.

13. Process according to claim 12, characterised in that the tablet cores are warmed to a temperature in the range from 35 to 60° C., preferably in the range from 40 to 55° C., before the spraying-on of the coating solution.

14. Process according to claim 12, characterised in that the tablets are dried for 10 to 20 minutes after the spraying-on of the coating solution.
